(19) United States
(12) Patent Application Publication
(10) Pub. No.: US 2024/0050434 A1
(43) Pub. Date: Feb. 15, 2024

(54) HORMAD1 THERAPEUTICS

(71) Applicants: Royal Cancer Hospital, London (GB); Breast Cancer Now, London (GB)

(72) Inventors: Andrew Tutt, London (GB); Callum Walker, London (GB); Daniel Weekes, London (GB); Jon Pines, London (GB); Christopher Lord, London (GB)

(21) Appl. No.: 18/266,190

(22) PCT Filed: Dec. 9, 2021
(86) PCT No.: PCT/EP2021/085027

§ 371 Date: Jun. 8, 2023

(30) Foreign Application Priority Data
Dec. 9, 2020 (GB) 2019375.1

(51) Int. Cl. A61K 31/522; C12Q 1/6886; A61K 31/506; A61K 31/519; A61K 31/4439; A61K 31/517; A61P 35/00

(52) U.S. Cl. CPC A61K 31/522; C12Q 1/6886; A61K 31/506; A61K 31/519; A61K 31/4439; A61K 31/517; A61P 35/00; C12Q 2600/106; C12Q 2600/158

(57) ABSTRACT

The present invention relates to the treatment of cancer and, in particular to the treatment of patients whose cancer expresses HORMAD1 with an agent that modulates mitotic processes, and diagnostic methods thereof.

### FIELD OF THE INVENTION

The present invention relates to the treatment of cancer and, in particular to the treatment of patients whose cancer expresses HORMAD1 with an agent that modulates mitotic processes; and diagnostic methods thereof.

### BACKGROUND OF THE INVENTION

Cancer is one of the most life threatening diseases. Cancer is a condition in which cells in a part of the body experience out-of-control growth. According to latest data from American Cancer Society, it is estimated that more than 1.7 million new cases of cancer would be diagnosed in the USA in 2019. Cancer is the second leading cause of death in the United States (second only to heart disease) and will claim more than 606,000 lives in 2019. In fact, it is estimated the average lifetime risk of developing cancer is 39% for American males and 38% for American women. Therefore, cancer constitutes a major public health burden and represents a significant cost in the United States. These figures are reflected elsewhere across most countries globally, although the types of cancer and relative proportions of the population developing the cancers vary depending upon many different factors including genetics and diet.

For decades, surgery, chemotherapy, and radiation were the established treatments for various cancers. Patients usually receive a combination of these treatments depending upon the type and extent of their disease. But chemotherapy is the most important option for cancer patients when surgical treatment (i.e. the successful removal of all diseased tissue) is not possible. While surgery is sometimes effective in removing tumours located at certain sites, for example, in the breast, colon, and skin, it cannot be used in the treatment of tumours located in other areas, such as the backbone, nor in the treatment of disseminated hematological cancers including cancers of the blood and blood-forming tissues (such as the bone marrow). In addition, chemotherapy is often used to supplement surgery to treat any diseased tissue that remained in the patient following surgery. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. One of the main causes of failure in chemotherapy is the development of drug resistance by the cancer cells, a serious problem that may lead to recurrence of disease or even death. Chemotherapy can also cause side effects as the drugs can also affect normal healthy cells as well as cancerous cells. For these and other reasons there remains a need for more effective chemotherapeutic options for treating cancers.

One area of exploration is personalised or precision medicine, where treatments can be selected based on a genetic understanding of the disease. Personalised medicine is a form of medicine that uses information about a person's own genes or proteins to inform a treatment strategy. In cancer, personalized medicine uses specific information about a person's tumor to help make a diagnosis, plan treatment, find out how well treatment is working, or make a prognosis. Examples of personalized medicine include using targeted therapies to treat specific types of cancer cells, such as HER2-positive breast cancer cells, or using tumor marker testing to help diagnose cancer.

Through the identification of new-targeted cancer therapies it may be possible to develop treatments which are more efficacious and/or exhibit reduced side effects in comparison to traditional chemotherapy options.

### SUMMARY OF THE INVENTION

In a first aspect of the present invention there is provided an agent that modulates mitotic processes for use in a method of treating a patient with cancer; said treatment comprising:
1. determining whether the cancer expresses HORMAD1; and, if so
2. administering to said patient an agent that modulates mitotic processes.

By targeting patients with cancers that express HORMAD1, we show remarkable sensitivity to agents that modulate mitotic processes. This targeted therapy provides advantageous efficacy and/or a reduction in side effects.

In a further aspect of the present invention there is provided an agent that modulates mitotic processes for use in a method of treating a patient with cancer; said treatment comprising:
1. determining whether a test sample from the patient expresses HORMAD1; and, if so
2. administering to said patient an agent that modulates mitotic processes.

In a further aspect of the present invention there is provided a method of treating a patient with cancer, said method comprising:
1. determining whether said cancer expresses HORMAD1; and, if so
2. administering to said patient an agent that modulates mitotic processes.

In a further aspect of the present invention there is provided a method of treating a patient with cancer, said method comprising:
1. determining whether a test sample from said patient has elevated levels of HORMAD1; and, if so
2. administering to said patient an agent that modulates mitotic processes.

In a further aspect of the present invention there is provided an in-vitro method for identifying an individual with cancer having suitability for treatment with an agent that modulates mitotic processes, said method comprising determining whether a sample from said individual expresses HORMAD1.

The present invention allows for the first time for a reliable diagnostic method to determine whether a patient could benefit from an agent that modulates mitotic processes. Such an approach allows healthcare professionals to select patients who will most benefit from these treatment options.

In a further aspect of the present invention there is provided an in-vitro method for identifying an individual with cancer having suitability for treatment with an agent that modulates mitotic processes; said method comprising identifying whether spindle assembly checkpoint defects are present during mitosis; where the presence of spindle assembly checkpoint defects indicates sensitivity to agents that modulate mitotic processes.

Monitoring the presence of spindle assembly checkpoint defects during mitosis provides an alternative diagnostic mechanism to identify patients who may be particularly sensitive to agents that modulate mitotic processes. This may or may not be patients who express HORMAD1.

In a further embodiment of the methods of the invention, the agent that modulates mitotic processes may be administered with a further agent. Preferably, the further agent is administered concurrently, sequentially or separately to the agent that modulates mitotic processes. In one embodiment, the further agent may be a microtubule stabiliser such as but not limited to paclitaxel or docetaxel or microtubule disrupter, such as but not limited to Eribulin, Vinorelbine, xabepilone or maytansine/MMAE linked antibody drug conjugate (ADC). In another embodiment, the further agent is a DNA replication stress and break inducing drug such as but not limited to a PARP inhibitor, platinum salt or topoisomerase inhibitor such a deruxtecan linked ADC.

In a further aspect of the present invention there is provided an agent that modulates mitotic processes for use in the treatment of a HORMAD1 positive cancer.

For the first time, it has been identified that agents that modulate mitotic processes are useful in the treatment of these cancers.

In another aspect of the invention, there is provided an agent that modulates mitotic processes for use in the treatment of cancer, preferably a HORMAD1-positive cancer, wherein the agent is used in combination with one or more further agents. Preferably, the further agent is administered concurrently, sequentially or separately to the agent that modulates mitotic processes. In one embodiment, the further agent may be a microtubule disrupter, such as but not limited to Eribulin, Vinorelbine and Ixabepilone. In another embodiment, the further agent may be a microtubule stabiliser, such as paclitaxel or docetaxel. In another embodiment the further agent is a DNA replication stress and break inducing drug such as but not limited to a PARP inhibitor, platinum salt or topoisomerase inhibitor such a deruxtecan linked ADC.

The following features apply to all aspects of the invention.

Cancers which express HORMAD1 (i.e. HORMAD1 positive cancers) include breast cancers, such as triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

In one embodiment, there is provided an agent that that modulates mitotic processes for use in the treatment of a HORMAD1 positive cancer, wherein the cancer is not breast, melanoma, colorectal, germ cell tumours of the testis or cancers of the bladder, cervix, or ovary. In a further embodiment, the cancer is leukaemia, sarcoma, uveal melanoma, cholangiocarcinoma, germ cell tumours of the testis, cancer of the oesophagus, head and neck cancer, pancreatic cancer, stomach cancer, thyroid cancer or cancer of the uterus.

The agent that modulates mitotic processes may be a spindle assembly checkpoint inhibitor.

The agent that modulates mitotic processes may be a BUB1 inhibitor.

The agent that modulates mitotic processes may be a MPSI inhibitor.

The agent that modulates mitotic processes may be an AURORA kinase inhibitor.

The agent that modulates mitotic processes may be an AURORA A kinase inhibitor.

The agent that modulates mitotic processes may be an AURORA B kinase inhibitor.

The agent that modulates mitotic processes may be a MASTL inhibitor.

The agent that modulates mitotic processes may be a PLK1 inhibitor.

The agent that modulates mitotic processes may be an inhibitor of cytokinesis. In one embodiment, the inhibitor may be selected from crizotinib and foretinib.

Determining whether the cancer expresses HORMAD1 or whether a test sample from the patient expresses HORMAD1, may involve identifying in a patient sample levels of, or the presence of, HORMAD1 gene amplification, HORMAD1 mRNA transcript and/or HORMAD1 protein expression. Thus, HORMAD1 gene amplification; HORMAD1 mRNA transcript levels; and/or HORMAD1 protein levels can be used as a diagnostic indicator for sensitivity to agents that modulate mitotic processes. In a further embodiment, determining whether the cancer expresses HORMAD1 may involve determining the level of HORMAD1 gene amplification, HORMAD1 mRNA transcript levels and/or HORMAD1 protein expression in a control, such as a patient without cancer or a HORMAD1 expressing cancer or a control sample or cell line therefrom, wherein HORMAD1 gene amplification, an increase in HORMAD1 mRNA transcript levels and/or HORMAD1 protein expression compared to the control is indicative of a cancer that expresses HORMAD1.

### DESCRIPTION OF THE FIGURES

FIG. 1a shows HORMAD1 RNA expression across a range of normal tissues; and 1b/c shows HORMAD1 gene expression in multiple cancers but not normal somatic tissues;

FIG. 2 shows the results of an siRNA screen for HORMAD1 expressing RPE1 cells. 2a shows doxycycline-inducible HORMAD1 gene expression construct viability; FIG. 2b shows a diagram illustrating the siRNA screen; 2c shows the results of the screen as the normalised percent inhibition (NPI) from doxycycline treated cells subtracted by the NPI of vehicle treated cells; and 2d shows a bar chart depicting 5 replicates for two BUB1 siRNA particles identified in the siRNA screen;

FIG. 3 shows the effect of BUB1 inhibitor, BAY-1816032, on HORMAD1 expressing cells in 2D culture;

FIG. 4 shows the effect of BUB1 inhibitor, BAY-1816032, on 3 HORMAD1-positive triple-negative breast cancer cell-lines (TNBC) and 2 HORMAD1-negative non-transformed breast cell-lines;

FIG. 5 shows the effect of BAY-1816032, in 3D spheroids and in genetically-engineered mouse model-derived tumors;

FIG. 6 shows further experiments with BAY-1816032 in HORMAD1-positive and -negative tumor models;

FIG. 7 shows the effect of HORMAD1 expression on MPSI inhibitor BOS172722 in RPE1 and SUM159 cells;

FIG. 8 shows the effect of HORMAD1 expression on AURORA A inhibitor MK-5108 in RPE1 cells;

FIG. 9 shows the effect of HORMAD1 expression on AURORA B inhibitor AZD1152-HQPA in RPE1 and SUM159 cells;

FIG. 10 shows 3D organoid experiments using AURORA B inhibitor AZD1152-HQPA in HORMAD1-positive and -negative tumour models;

FIG. 11 shows the effect of HORMAD1 expression on spindle assembly checkpoint defects and mitotic segregation errors;

FIG. 12 shows the effects of combinations in HORMAD1 positive tumour models.

FIG. 13 shows that HORMAD1 perturbs the spindle assembly checkpoint. A. Diagram of experimental outline. Cells were exposed to doxycycline for 3 days, arrested in mitosis using 300 nM nocodazole or 100 nM Paclitaxel. Mitotic cells were filmed every 4 minutes for 1200 minutes, and the time cells remained arrested in mitosis before exiting via mitotic slippage was assessed. B. Curve indicating premature mitotic slippage following nocodazole exposure in RPE1 cells induced to express HORMAD1. RPE1 cells expressing a doxycycline-inducible HORMAD1 cDNA expression construct were exposed to doxycycline or vehicle (H₂O) for three days at which point cells were exposed to 300 nM nocodazole and filmed every 4 minutes using time-lapse microscopy. The time individual cells remained arrested in mitosis was quantified and presented as the % of cells arrested in mitosis at each time point. Data reflects a total of 125 cells from 3 biological repeats and was analysed using Mantel-Cox test. C. Curve indicating premature mitotic slippage following nocodazole exposure in RPE1 cells induced to express HORMAD1. RPE1 cells expressing a doxycycline-inducible HORMAD1 cDNA expression construct were exposed to doxycycline or vehicle (H₂O) for three days at which point cells were exposed to 100 nM paclitaxel and filmed every 4 minutes using time-lapse microscopy. The time individual cells remained arrested in mitosis was quantified and presented as the % of cells arrested in mitosis at each time point. Data reflects a total of 125 cells from 3 biological repeats and was analysed using Mantel-Cox test. D. Western blot illustrating HORMAD1 depletion in H1299 cells expressing two individual shRNA particles that target HORMAD1. H1299 cells expressing two individual, doxycycline-inducible, shRNA particles targeting HORMAD1 as well as a non-targeting control were grown for three days in the presence of doxycycline at which point cell lysates were generated and analysed by SDS-PAGE and western blotting using anti-HORMAD1 and anti-β-ACTIN antibodies. E. Curve indicating delayed mitotic slippage following nocodazole exposure in H1299 cells depleted of HORMAD1. H1299 cells expressing two individual, doxycycline-inducible, shRNA particles targeting HORMAD1 as well as a non-targeting control were grown for three days in the presence of doxycycline at which point cells were exposed to 300 nM nocodazole and filmed every 4 minutes using time-lapse microscopy. The time individual cells remained arrested in mitosis was quantified and presented as the % of cells arrested in mitosis at each time point. Data reflects a total of 90 cells from 3 biological repeats and was analysed using Mantel-Cox test with Bonferonni correction. F. Diagram of experimental outline. Cells were exposed to doxycycline for 3 days, arrested in mitosis using 300 nM nocodazole for 6 hours, before being treated with the indicated dose of MPSI inhibitor. Mitotic cells were filmed every 2 minutes for 400 minutes, and the time taken before cells exited mitosis via mitotic slippage was assessed. G. Curve indicating premature mitotic slippage following nocodazole and reversine exposure in RPE1 cells induced to express HORMAD1. RPE1 cells expressing a doxycycline-inducible HORMAD1cDNA expression construct were exposed to doxycycline or vehicle (H₂O) for three days at which point cells were exposed to 300 nM nocodazole for 6 hours and then 100 nM and 200 nM reversine or vehicle (DMSO) before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. H. Curve indicating premature mitotic slippage following nocodazole and reversine exposure in RPE1 cells induced to express HORMAD1. RPE1 cells expressing a doxycycline-inducible HORMAD1 cDNA expression construct were exposed to doxycycline or vehicle (H₂O) for three days at which point cells were exposed to 300 nM nocodazole for 6 hours and then 50 nM BOS172722 or vehicle (H₂O) before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. I. Graph illustrating increased centromere containing micronuclei in HORMAD1 expressing RPE1 cells exposed to a MPSI inhibitor. RPE1 cells expressing a doxycycline-inducible HORMAD1 expression construct were exposed to doxycycline or vehicle (H₂O) and were exposed to 200 nM of the MPSI inhibitor, BOS172722. 3 days post drugging, immunocytochemistry was performed using anti-centromere antibodies alongside DAPI. The % of cells with micronuclei was quantified from >1000 cells across 3 biological replicates and was analysed using Student's t-test with Bonferroni correction with Bonferonni correction. J. Curve indicating delayed mitotic slippage following nocodazole and reversine exposure in H1299 cells depleted of HORMAD1. H1299 cells expressing two individual, doxycycline-inducible, shRNA particles targeting HORMAD1 as well as a non-targeting control were grown for three days in the presence of doxycycline at which point cells were exposed to 300 nM nocodazole for 6 hours and then 200 nM reversine and filmed every 2 minutes using time-lapse microscopy. The time individual cells remained arrested in mitosis was quantified and presented as the % of cells arrested in mitosis at each time point. Data reflects a total of 60 cells from 2 biological repeats and was analysed using Mantel-Cox test with Bonferonni correction;

FIG. 14 shows HORMAD1 drives cellular sensitivity to MPSI and Aurora B inhibition A. Growth curve indicating decreased cellular fitness in HORMAD1 expressing RPE1 cells treated with an MPSI inhibitor. RPE1 cells expressing a doxycycline-inducible HORMAD1 expression construct were exposed to doxycycline or vehicle (H₂O) and were exposed to 200 nM of the MPSI inhibitor, BOS172722. Cell confluence was analysed every 6 hours for 10 days, using incucyte software. Data are from 3 biological replicates and analysed using a two-way ANOVA. B. Growth curve indicating increased decreased cellular fitness in HORMAD1 expressing RPE1 cells treated with an Aurora B inhibitor. RPE1 cells expressing a doxycycline-inducible HORMAD1 expression construct were exposed to doxycycline or vehicle (H₂O) and were exposed to 20 nM of the Aurora B inhibitor, AZD1152-HQPA. Cell confluence was analysed every 6 hours for 10 days, using incucyte software. Data are from 3 biological replicates and analysed using a two-way ANOVA. HORMAD1 drives SAC perturbations C. Clonogenic survival assay indicating increased MPSI inhibitor sensitivity in HORMAD1 expressing RPE1 cells. RPE1 cells expressing a doxycycline-inducible HORMAD1 expression construct were exposed to doxycycline or vehicle (H₂O) and to increasing doses of the MPSI inhibitor, BOS172722. 7-10 days post drugging, cells were fixed, stained with SRB, and colonies were counted using Cell Profiler. Data show 3 technical replicates, normalised to DMSO treatment, and representative of 3 biological replicas and analysed using a two-way ANOVA. D. Clonogenic survival assay indicating increased MPSI inhibitor sensitivity in HORMAD1 expressing SUM159 cells. SUM159 cells expressing a doxycycline-inducible HORMAD1 expression construct were exposed to doxycycline or vehicle (H₂O) and to increasing doses of the MPSI inhibitor, BOS172722. 10 days post drugging, cells were fixed, stained with SRB, and colonies were counted using Cell Profiler. Data show 3 technical replicates, normalised to DMSO treatment, and representative of 3 biological replicas and analysed using a two-way ANOVA. E. Clonogenic survival assay indicating increased Aurora B inhibitor sensitivity in HORMAD1 expressing RPE1 cells. RPE1 cells expressing a doxycycline-inducible HORMAD1 expression construct were exposed to doxycycline or vehicle (H₂O) and to increasing doses of the Aurora B inhibitor, AZD1152-HQPA. 7-10 days post drugging, cells were fixed, stained with SRB, and colonies were counted using Cell Profiler. Data show 3 technical replicates, normalised to DMSO treatment, and representative of 3 biological replicas and analysed using a two-way ANOVA. F. Clonogenic survival assay indicating increased Aurora B inhibitor sensitivity in HORMAD1 expressing SUM159 cells. SUM159 cells expressing a doxycycline-inducible HORMAD1 expression construct were exposed to doxycycline or vehicle (H₂O) and to increasing doses of the Aurora B inhibitor, AZD1152-HQPA. 10 days post drugging, cells were fixed, stained with SRB, and colonies were counted using Cell Profiler. Data show 3 technical replicates, normalised to DMSO treatment, and representative of 3 biological replicas and analysed using a two-way ANOVA;

FIG. 15 shows HORMAD1 expression induces cellular sensitivity to BUB1 inhibition A. Western blot illustrating efficient BUB1knock-down in RPE1 cells transfected with 2 individual siRNA particles. RPE1 cells were reverse transfected with BUB1 or control siRNA particles for 3 days. Cell lysates were collected, and analysed by western blotting using anti-BUB1 and anti-β-ACTIN antibodies. B. Graph indicating reduced cellular survival in HORMAD1 expressing RPE1 cells depleted of BUB1 using siRNA. RPE1 cells expressing a doxycycline-inducible HORMAD1 expression construct were exposed to doxycycline or vehicle, and were reverse transfected with BUB1 or control siRNA particles. 5 days post transfection, cellular viability was estimated using cell titer glo reagent and surviving fractions were calculated relative to DMSO treated cells. These data are representative HORMAD1 drives SAC perturbations of 3 biological replicas, each with 5 technical replicates per replicate. Statistical significance estimated using a one-way ANOVA with Sidak's correction. C. Western blot illustrating expression of HORMAD1 in wild-type RPE1 cells and RPE1 cells depleted of BUB1 using CRISPR. WT and BUB1 depleted RPE1 cells expressing a doxycycline-inducible HORMAD1 expression construct, were exposed to doxycycline or vehicle for 3 days. Cell lysates were collected, and analysed by SDS-PAGE and western blotting using anti-HORMAD1, anti-H3 and anti-BUB1 antibodies. D. Graph indicating reduced cellular survival in HORMAD1 expressing RPE1 cells depleted of BUB1 using CRISPR. WT and BUB1 depleted RPE1 cells expressing a doxycycline-inducible HORMAD1 expression construct were exposed to doxycycline or vehicle. 7-10 days post doxycycline induction, cells were fixed, stained with SRB, and cellular viability was estimated using SRB absorbance readings. These data are representative of 3 biological replicas, each with 3 technical replicates per replicate. Statistical significance estimated using a one-way ANOVA with Sidak's correction. E. Clonogenic survival assay indicating increased cellular sensitivity to a BUB1 inhibitor following HORMAD1 expression. RPE1 expressing a doxycycline-inducible HORMAD1 expression construct, were exposed to doxycycline or vehicle. Cells were exposed to increasing doses of the BUB1 inhibitor, BAY-1816032, for 7-10 days. Cells were fixed, stained with SRB, and cellular viability was estimated using SRB absorbance readings. Data show 3 technical replicates, normalised to DMSO treatment, and representative of 3 biological replicas. Statistical significance estimated using a two-way ANOVA, P<0.0001. F. Growth curve indicating increased decreased cellular fitness in HORMAD1 expressing RPE1 cells treated with a BUB1 inhibitor. RPE1 cells expressing a doxycycline-inducible HORMAD1 expression construct were exposed to doxycycline or vehicle (H2O) and were exposed to 3 μM of the BUB1 inhibitor, BAY-1816032, for 14 days. Cells were imaged every 4 hours, and confluence was continually measured using incucyte software. Growth curves were plotted, based on confluence. Data represent 3 biological replicas. Statistical significance estimated using a two-way ANOVA, P<0.0001 with Bonferonni correction. G. Western blot illustrating bimodal HORMAD1 expression in breast cancer patient-derived organoids model systems. Patient-derived organoids models were grown in culture and cell lysates collected and analysed using SDS-PAGE and western blotting using anti-HORMAD1 and anti-β-ACTIN antibodies. H. Dose response curves illustrating increased BUB1 inhibitor sensitivity in HORMAD1-positive patient-derived organoid models. Patient-derived organoids models were exposed to increasing doses of the BUB1 inhibitor, BAY-1816032, for 14 days. Cellular viability was estimated using cell titer glo reagent and surviving fractions were calculated relative to DMSO treated cells. These data are representative of 3 biological replicas, each with 3 technical replicates. Left, dose response curves for each organoid. Right, area under the curve analysis based on HORMAD1 status. Statistical significance estimated using a Student's t-test. I. HORMAD1 binds to Aurora B in mitosis, impeding its interaction with INCENP and in turn the phosphorylation of its substrates. HORMAD1-positive cells therefore have a weakened SAC and are then more sensitive to BUB1, Aurora B and MPSI inhibitors;

FIG. 16 shows that HORMAD1 drives sensitivity to additional MPSI and Aurora kinase inhibitors. A. Clonogenic survival assay indicating increased MPSI inhibitor sensitivity in HORMAD1 expressing RPE1 cells. RPE1 cells expressing a doxycycline-inducible HORMAD1 expression construct were exposed to doxycycline or vehicle (H₂O) and to increasing doses of the MPSI inhibitor, AZ-3146. 7-10 days post drugging, cells were fixed, stained with SRB, and colonies were counted using Cell Profiler. Data shows 3 technical replicates, normalised to DMSO treatment, and representative of 3 biological replicas and analysed using a two-way ANOVA. B. Clonogenic survival assay indicating increased Aurora inhibitor sensitivity in HORMAD1 expressing RPE1 cells. RPE1 cells expressing a doxycycline-inducible HORMAD1 expression construct were exposed to doxycycline or vehicle (H2O) and to increasing doses of the Aurora inhibitor, MK-5108. 7-10 days post drugging, cells were fixed, stained with SRB, and colonies were counted using Cell Profiler. Data shows 8 technical replicates, normalised to DMSO treatment, and representative of 3 biological replicas and analysed using a two-way ANOVA;

FIG. 17 shows HORMAD1 causes SAC perturbations in multiple models. A. Western blot illustrating HORMAD1 depletion in HeLa cells transfected with siRNA particles that target HORMAD1. HeLa cells were transfected with two individual siRNA particles targeting HORMAD1 as well as a non-targeting control, grown for three days at which point cell lysates were generated and analysed SDS-PAGE and western blotting using anti-HORMAD1 and anti-H3 antibodies. B. Curve indicating delayed mitotic slippage following nocodazole exposure in HeLa cells depleted of HORMAD1. HeLa cells were transfected with two individual siRNA particles targeting HORMAD1 as well as a non-targeting control, grown for three days at which point cells were exposed to 300 nM nocodazole and filmed every 4 minutes using time-lapse microscopy. The time individual cells remained arrested in mitosis was quantified and presented as the % of cells arrested in mitosis at each time point. Data reflects a total of 125 cells from 3 biological repeats and was analysed using Mantel-Cox test with Bonferonni correction. C. Western blot illustrating HORMAD1 depletion in MDA-MB-436 cells expressing two individual shRNA particles that target HORMAD1. MDA-MB-436 cells expressing two individual, doxycycline-inducible, shRNA particles targeting HORMAD1 as well as a non-targeting control were grown for three days in the presence of doxycycline at which point cell lysates were generated and analysed SDS-PAGE and western blotting using anti-HORMAD1 and anti-6-ACTIN antibodies. D. Curve indicating delayed mitotic slippage following nocodazole exposure in MDA-MB-436 cells depleted of HORMAD1. MDA-MB-436 cells expressing two individual, doxycycline-inducible, shRNA particles targeting HORMAD1 as well as a non-targeting control were grown for three days in the presence of doxycycline at which point cells were exposed to 300 nM nocodazole and filmed every 4 minutes using time-lapse microscopy. The time individual cells remained arrested in mitosis was quantified and presented as the % of cells arrested in mitosis at each time point. Data reflects a total of 50 cells from 2 biological repeats and was analysed using Mantel-Cox test with Bonferonni correction. E. Western blot illustrating doxycycline-induced HORMAD1 (HA tagged) in MCF10A cells alongside endogenous HORMAD1 expression in MDA-MB-436 cells. MCF10A cells expressing a doxycycline-inducible HORMAD1 cDNA expression construct were exposed to doxycycline or vehicle (H₂O) for three days at which point cell lysates were generated and analysed by SDS-PAGE and western blotting using anti-HORMAD1 and anti-β-ACTIN antibodies. F. Curve indicating premature mitotic slippage following nocodazole and reversine exposure in MCF10A cells induced to express HORMAD1. MCF10A cells expressing a doxycycline-inducible HORMAD1 cDNA expression construct were exposed to doxycycline or vehicle (H2O) for three days at which point cells were exposed to 300 nM nocodazole for 6 hours and then 100 nM reversine or vehicle (DMSO) before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. G. Western blot illustrating doxycycline-induced HORMAD1 in SUM159 cells alongside endogenous HORMAD1 expression in MDA-MB-436 cells. SUM159 cells expressing a doxycycline-inducible HORMAD1 cDNA expression construct were exposed to doxycycline or vehicle (H2O) for three days at which point cell lysates were generated and analysed by SDS-PAGE and western blotting using anti-HORMAD1 and anti-β-ACTIN antibodies. H. Curve indicating premature mitotic slippage following nocodazole and reversine exposure in SUM159 cells induced to express HORMAD1. RPE1 cells expressing a doxycycline-inducible HORMAD1 cDNA expression construct were exposed to doxycycline or vehicle (H2O) for three days at which point cells were exposed to 300 nM nocodazole for 6 hours and then 100 nM reversine or vehicle (DMSO) before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. I. Curve indicating delayed mitotic slippage following nocodazole and reversine exposure in HeLa cells depleted of HORMAD1. HeLa cells were transfected with two individual siRNA particles targeting HORMAD1 as well as a non-targeting control, grown for three days at which point cells were exposed to 300 nM nocodazole for 6 hours and then 100 nM reversine or vehicle (DMSO) and filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. J. Curve indicating delayed mitotic slippage following nocodazole and reversine exposure in MDA-MB-436 cells depleted of HORMAD1. MDA-MB-436 cells expressing two individual, doxycycline-inducible, shRNA particles targeting HORMAD1 as well as a non-targeting control were grown for three days in the presence of doxycycline at which point cells were exposed to 300 nM nocodazole for 6 hours and then 200 nM reversine or vehicle (DMSO) and filmed every 2 minutes using time-lapse microscopy. The time individual cells remained arrested in mitosis was quantified and presented as the % of cells arrested in mitosis at each time point. Data reflects a total of 75 cells from 3 biological repeats and was analysed using Mantel-Cox test with Bonferonni correction;

FIG. 18 shows the HORMAD1 positive organoid KCL625 is high sensitivity to Aurora B (AZD1152-HQPA) (A) and MPSI (BOS172722) inhibition (B). Organoids were treated with the indicated doses of drug for 10 days and cell viability assessed by cell titer glo. Data is presented as mean and standard deviation of triplicate wells;

FIG. 19 shows expression of the HORMA domain of HORMAD1 impairs mitotic arrest. RPE1 cells expressing a doxycycline-inducible Full length or HORMA domain only expression construct were exposed to doxycycline or vehicle for three days, at which point cells were exposed to 300 nM nocodazole for 6 hours and then 100 nM reversine, before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction.

### DETAILED DESCRIPTION OF THE INVENTION

In the present application, a number of general terms and phrases are used, which should be interpreted as follows.

In one embodiment, “HORMAD1” may be alternatively known as CT46 or DKFZP434A1315. The Ensembl version number may be ENSG00000143452.16. It may refer to a nucleic acid sequence comprising a sequence defined in NM_001199829.2 or NM_032132.5.

The term “treating”, as used herein, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above.

“Patient” includes humans, non-human mammals (e.g., dogs, cats, rabbits, cattle, horses, sheep, goats, swine, deer, and the like) and non-mammals (e.g., birds, and the like). Preferably, the patient is a human patient.

The present invention has identified that overexpression of HORMAD1 in cancer sub-types can be exploited by agents that modulate mitotic processes to create targeted cancer therapies.

We have found that HORMAD1 causes defects in the SAC (spindle assembly checkpoint), which then creates a high dependency towards normal mitotic processes that would otherwise be better tolerated in tumour cells with a functional SAC.

By “agent that modulates mitotic processes” is meant any agent that modulates, preferably disrupts, impairs or inhibits one or more mitotic process. Mitotic kinases, including but not limited to MPSI, Aurora B, and BUB1, play diverse roles in the mitotic processes. For instance, Aurora B regulates spindle assembly, the spindle assembly checkpoint, cytokinesis/abscission checkpoint as well as error-correction and the G2/M transition checkpoint. MPSI is the master regulator of the SAC, but also plays well-defined roles in spindle assembly and error-correction. BUB1 kinase regulates the spindle assembly checkpoint by potentiating MPSI activity, but also regulates Aurora B activity through-out mitosis, as well as regulating chromosome congression via Aurora B independent mechanisms. These examples demonstrate the intertwined nature of mitotic processes and how inhibitors of a given mitotic process would also have direct or indirect effects on other mitotic processes.

Accordingly, in a preferred embodiment, such an agent modulates at least one and preferably disrupts, impairs or inhibits, at least one mitotic process including but not limited to spindle assembly, chromosomal alignment, chromosome segregation and error correction, cytokinesis; or modulates (including impairing, deactivating or activating) the mitotic cell cycle checkpoints, G2/M, SAC (spindle assembly checkpoint) and (cytokinetic) abscission. In one embodiment, the agent modulates, preferably impairs a mitotic checkpoint, preferably the SAC or mitotic error correction. In a further embodiment, the agent is a mitotic checkpoint kinase inhibitor. In one embodiment, the agent that modulates at least one mitotic process is not a PARP inhibitor or cisplatin.

HORMAD1 is the mammalian homolog of HOPI, a meiotic HORMA-domain containing protein first identified in yeast. HORMAD1 regulates numerous aspects of meiotic cell behaviour, including chromosome homolog synapsis, the initiation and repair of SPO11-induced double-stranded DNA breaks, as well as the subsequent control over cell cycle checkpoints that permits the generation and maturation of gamete cells.

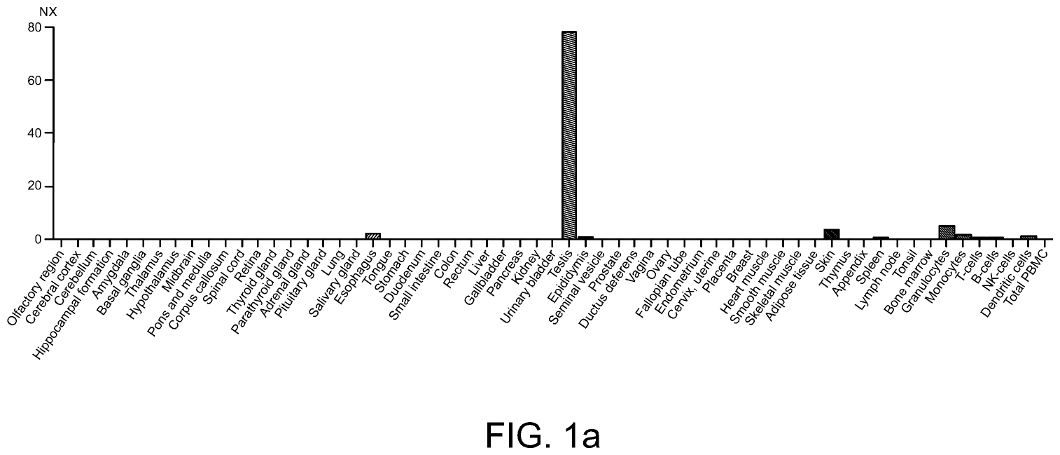

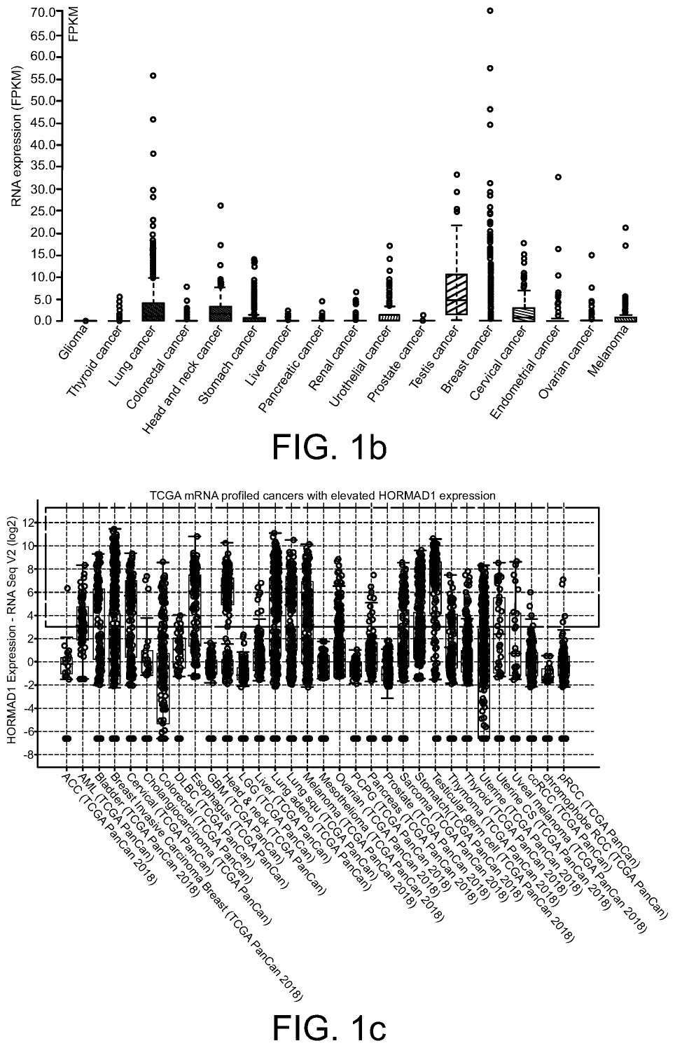

HORMAD1's normal physiological role and normal gene expression appears restricted to germ-line cells and HORMAD1 is not normally expressed in non-transformed somatic tissues. FIG. 1a shows a consensus data set from the human protein atlas (www.proteinatlas.orq, 20 Jan. 2020) for HORMAD1. Consensus normalized expression (NX) levels is shown for 55 tissue types and 6 blood cell types, created by combining the data from the three transcriptomics datasets (HPA, GTEx and FANTOM5). It can be seen that the testes represents the major expression tissue with minor expression in skin, granulocytes, monocytes and dendritic cells. HORMAD1 is therefore expressed strongly in testis, weakly in placenta (not shown in FIG. 1a), with expression in other tissues less than 1% of that seen in the testis.

Using tumour mRNA expression data from the same source, HORMAD1 expression in human cancers was assessed. The results are shown in FIG. 1b where it can be seen that HORMAD1 is expressed in a number of cancer histotypes. An analysis of tumour mRNA expression from large-scale tumour resequencing studies included in cbioportal (https://www.cbioportal.org) replicated this analysis and the results are shown in FIG. 1c. These results are also discussed in Uhlén et al., 2015 and Uhlén et al., 2017.

Thus, HORMAD1 is expressed in certain cancer sub-types, including but not limited to breast cancers, such as triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung (including small cell or non-small cell lung cancer), ovary, pancreas, stomach, thyroid and uterus.

In a preferred embodiment, the cancer is breast cancer, such as triple negative (ER, PgR HER2 negative) and/or basal like breast cancer.

It can therefore be seen that HORMAD1 is typically not expressed in normal tissue so an absence of HORMAD1 expression would be expected in normal, non-tumour, tissue. By contrast, the presence of HORMAD1 biomarkers—e.g. the expression of HORMAD1 in a sample is an effective diagnostic marker for certain cancers herein disclosed.

Accordingly, in one embodiment of the aspects described herein, the HORMAD1 positive cancer may be selected from breast cancer, preferably triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

By expression of HORMAD1 is meant RNA or protein expression. Accordingly, suitable biomarkers include: (i) HORMAD1 gene amplification (i.e. an increase in the number of copies of the HORMAD1 gene, preferably to two or above); (ii) presence of or elevated levels of HORMAD1 mRNA transcript; (iii) and/or presence of or elevated levels of HORMAD1 protein. Any one or all of these biomarkers can be used to detect HORMAD1-positive cancers. The expression of HORMAD1 may be detected by any suitable method.

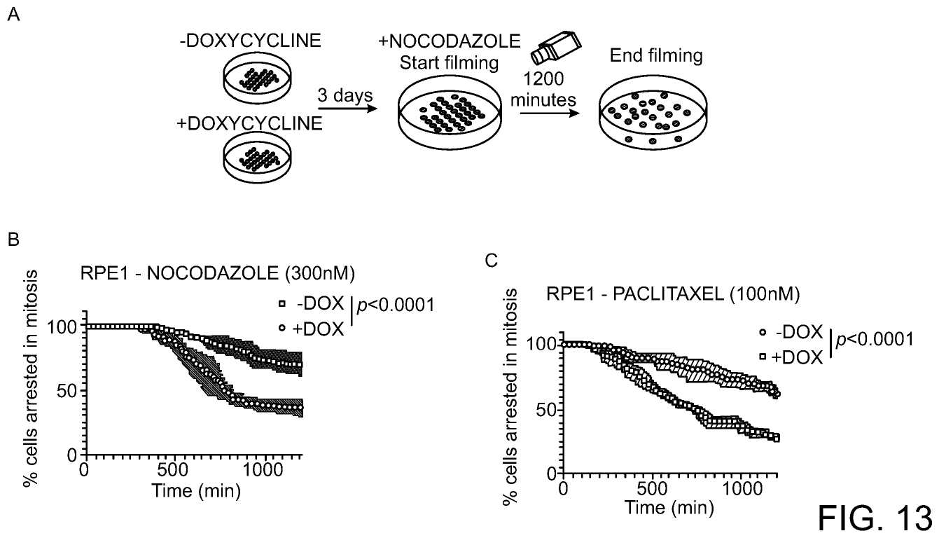

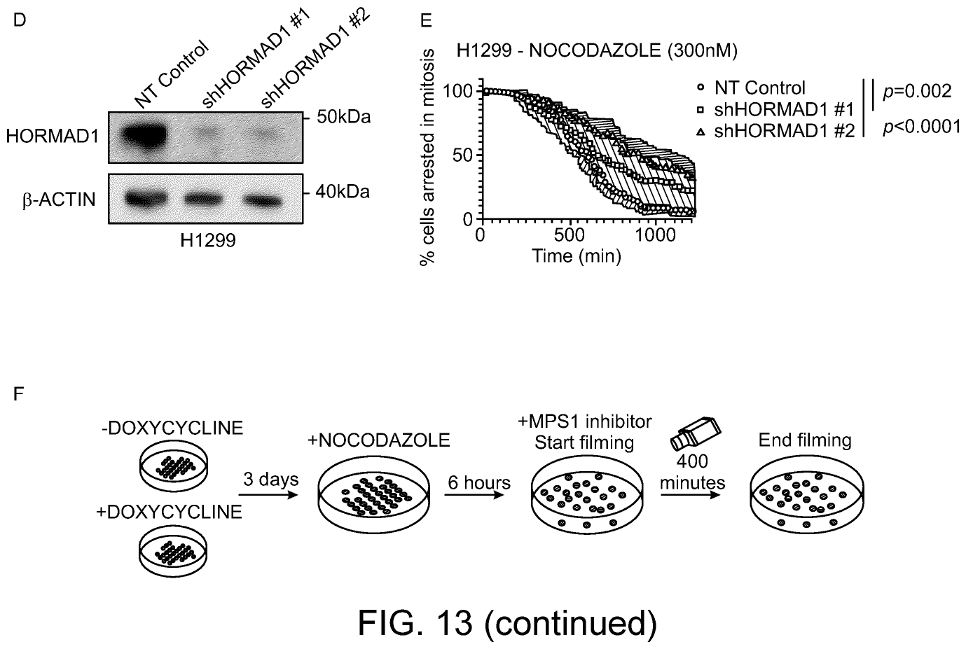

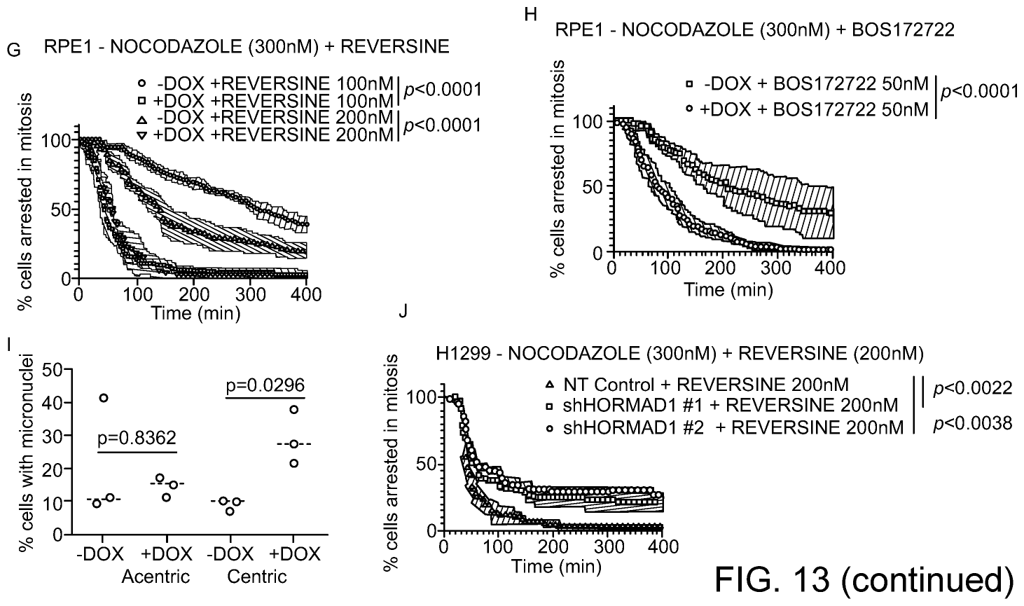

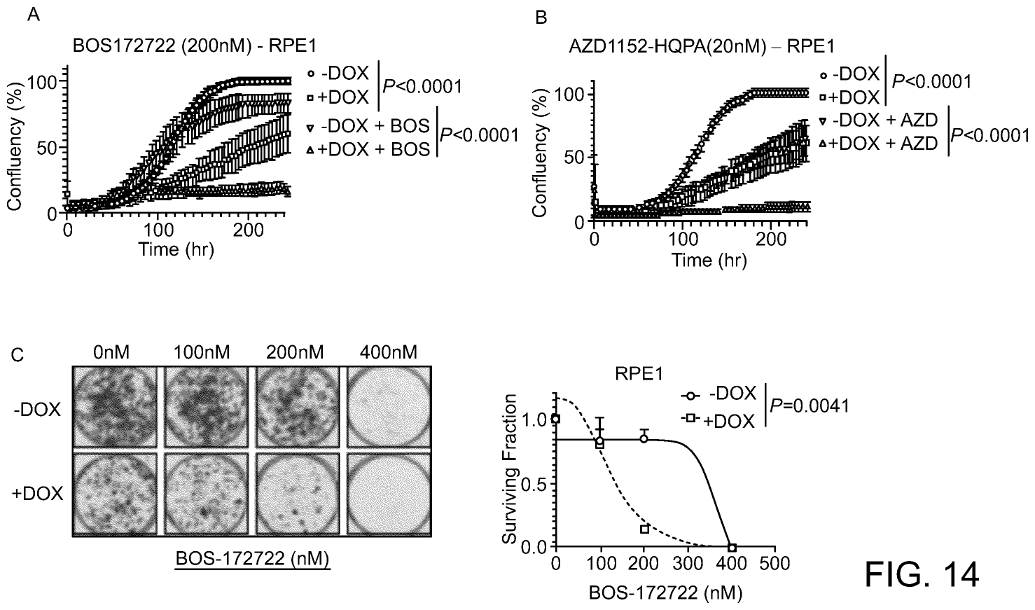

As shown in FIG. 13, we have found that the aberrant expression of HORMAD1 in tumour cells perturbs the ability of cells to delay mitosis in response to improperly attached kinetochores through a weakening of the SAC and/or error correction by a mechanism that is independent of MAD2L1. Cells with a defective SAC are unable to maintain mitotic arrest for prolonged periods of time and undergo mitotic slippage sooner than cells with a functional SAC.

HORMAD1 exerts these effects by binding to Aurora B, disrupting the association with its co-factor, INCENP, and impairing the phosphorylation of Aurora B substrates. Consistent with this mechanism, aberrant expression of HORMAD1 drives cell sensitivity to either clinical MPSI or Aurora kinase inhibitors and is synthetic lethal with depletion or small molecule inhibition of BUB1. BUB1 inhibition also sensitises patient-derived tumour organoids that over-express HORMAD1. In other words, we have shown that somatic cells that express HORMAD1 are hyper-dependent on the agents involved in mitosis, such as the agents mediating the SAC activation signal, including MPSI and any residual Aurora B kinase, for cell fitness, proliferation and clonal survival.

As such, taken together, these data suggest that the out-of-context expression of HORMAD1 in tumour cells drives a weakening of the processes that control mitotic fidelity that contributes to chromosomal instability and induces dependency on a number of clinically relevant therapeutic targets. As such, tumoral HORMAD1 expression can act as a patient selection biomarker for synthetic lethal sensitivity to BUB1, Aurora B or MPSI inhibitors.

Thus, the present invention has identified an unexpected link between HORMAD1 expression and tumour cell-specific sensitivity/synthetic lethality to agents involved in mitotic processes.

These include BUB1, MPSI, Aurora kinases, including AURKA, AURKB and/or AURKC, MASTL inhibitors, PLK1 inhibitors and inhibitors of cytokinesis. In each case, the synthetic lethal effects can be elicited using clinical small molecule (low molecular weight) kinase inhibitors, even when used in the presence of standard of care or emerging therapeutics.

BUB1 is a serine/threonine protein kinase that regulates numerous aspects of mitosis (Bolanos-Garcia & Blundell, 2011), including the regulation of chromosome congression, the spindle assembly checkpoint (SAC), and cytokinesis (Bolanos-Garcia & Blundell, 2011). The SAC (also known as the mitotic checkpoint) prevents the further progression of the cell cycle until each kinetochore, a protein complex associated with chromosomal centromeres to which the microtubule spindles attach, is attached to a spindle pole, thus preventing abnormalities in how the genetic material is divided between daughter cells (Musacchio, 2015). The SAC is activated by another serine/threonine kinase, MPSI (also known as TTK), which catalyses phosphorylation of MELT motifs on the KNL1 protein to generate docking sites for BUB1/BUB3 dimers (Primorac et al., 2013; Overlack et al., 2015). The BUB1/BUB3 dimers that form, together with CDC20, bind the active “closed” conformation of the MAD2 protein, which initiates the formation of the diffusible mitotic checkpoint complex (MCC); the MCC in turn inhibits the ubiquitin ligase APC/C, which prevents the onset of anaphase.

In part, the activity of BUB1 kinase in the control of mitosis is mediated by its ability to phosphorylate histone H2A present at the centromeric region of duplicated chromosomes; these histone phosphorylation events generate binding sites for the Shugoshin proteins (SGO) 1 and 2, which protect centromeric cohesin from premature degradation (Kawashima et al., 2010). Histone H2A phosphorylation by BUB1, in concert with Haspin-mediated histone H3 phosphorylation, also localises the chromosome passenger complex (CPC—consisting of INCENP, survivin, borealin, and another protein kinase, Aurora kinase B (AURKB)) to centromeres (Wang et al., 2011). Given this function, mouse embryonic fibroblasts (MEF) lacking BUB1 catalytic activity are unable to localise AURKB at the inner centromeres, exhibit reduced phosphorylation of centromeric substrates of AURKB, and are compromised in their ability to efficiently correct spindle attachment errors (Ricke et al., 2012). The failure to correctly localise AURKB because of BUB1 kinase inhibition compromises cells' ability to resolve spindle attachment errors and results in an increased rate of chromosome alignment defects, especially when these are caused by drugs that induce spindle attachment defects, such as the microtubule stabilizer paclitaxel (Baron et al., 2012).

It is believed that inhibition of kinases involved in the control of mitosis (e.g. BUB1, AURKA, AURKB or MPSI) can be used to target cancers by exacerbating existing defects in spindle function and the control of the SAC (Oser et a., 2019). For example, small molecule BUB1 inhibitors that inhibit the catalytic activity of BUB1 have been discovered that block cancer cell proliferation (Siemeister et al., 2019), as have MPSI inhibitors (Anderhub et al., 2019) and small molecule AURORA A (Shimomura et al., 2010) and AURORA B (Yang et al., 2007; Wilkinson et al., 2007, Oser et al., 2019).

In an embodiment, the agent that modulates mitotic processes of the present invention include BUB1 inhibitors, MPSI inhibitors, AURORA kinase inhibitors including AURORA A kinase inhibitors and AURORA B kinase inhibitors, MASTL inhibitors, PLK1 inhibitors and inhibitors of cytokinesis including but not limited to crizotinib and foretinib.

Suitable BUB1 inhibitors include BAY-1816032 (structure from Siemeister et al., 2019), BAY-320 and BAY-524. In a preferred embodiment the BUB1 inhibitor is BAY-1816032.

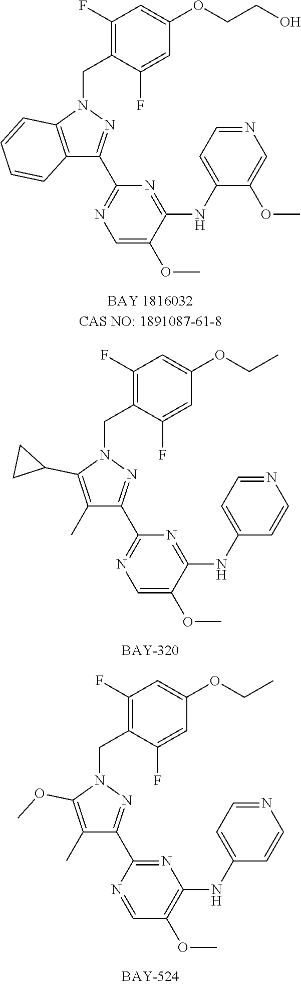

Suitable MPSI inhibitors include BOS172722 (structure from Woodward et al., 2018) AZ-3146, NMS-P153, CFI-402257, BAY-1217389, and BAY-1161909.

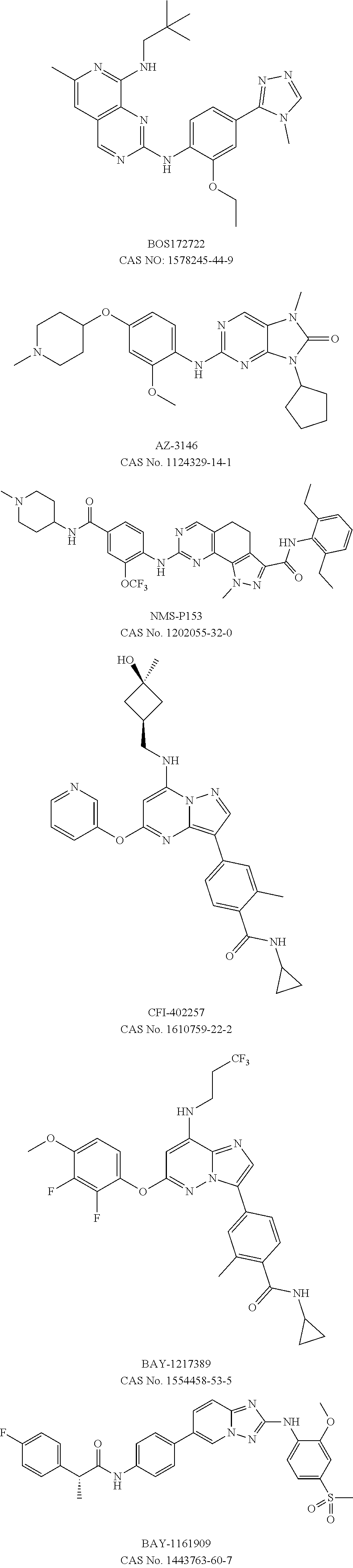 [SMILES: CCOc1cc(-c2nncn2C)ccc1Nc1ncc2cc(C)nc(NCC(C)(C)C)c2n1 ; COc1cc(OC2CCN(C)CC2)ccc1Nc1ncc2c(n1)n(C1CCCC1)c(=O)n2C ; CCc1cccc(CC)c1NC(=O)c1nn(C)c2c1CCc1cnc(Nc3ccc(C(=O)NC4CCN(C)CC4)cc3OC(F)(F)F)nc1-2 ; Cc1cc(-c2cnn3c(NC[C@H]4C[C@@](C)(O)C4)cc(Oc4cccnc4)nc23)ccc1C(=O)NC1CC1 ; COc1ccc(Oc2cc(NCCC(F)(F)F)c3ncc(-c4ccc(C(=O)NC5CC5)c(C)c4)n3n2)c(F)c1F ; COc1cc(S(C)(=O)=O)ccc1Nc1nc2ccc(-c3ccc(NC(=O)[C@H](C)c4ccc(F)cc4)cc3)cn2n1]

Suitable AURORA kinase inhibitors include AURORA A kinase inhibitors and AURORA B kinase inhibitors.

Suitable AURORA A kinase inhibitors includes MK-5108 (CAS No: 1010085-13-8), which inhibits AURORA A kinase activity at sub-nanomolar concentrations with ˜200-fold specificity over AURORA B and C (Shimomura et al., 2010).

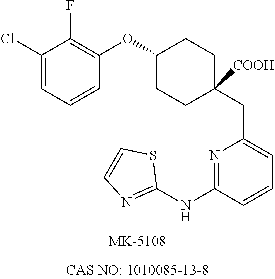 [SMILES: O=C(O)[C@]1(Cc2cccc(Nc3nccs3)n2)CC[C@@H](Oc2cccc(Cl)c2F)CC1]

Suitable AURORA B kinase inhibitors include AZD1152-HQPA (and AZD1152). The AZD1152 prodrug is rapidly converted into AZD1152-HQPA (CAS NO: 722544-51-6) in plasma and potently inhibits AURORA B kinase activity via competitive ATP inhibition (Mortlock et al., 2007).

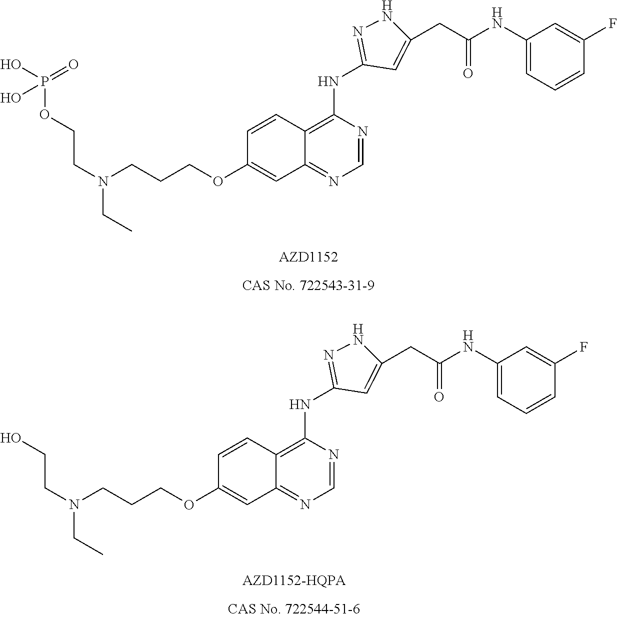 [SMILES: CCN(CCCOc1ccc2c(Nc3cc(CC(=O)Nc4cccc(F)c4)[nH]n3)ncnc2c1)CCOP(=O)(O)O ; CCN(CCO)CCCOc1ccc2c(Nc3cc(CC(=O)Nc4cccc(F)c4)[nH]n3)ncnc2c1]

Further Aurora kinase inhibitors include:

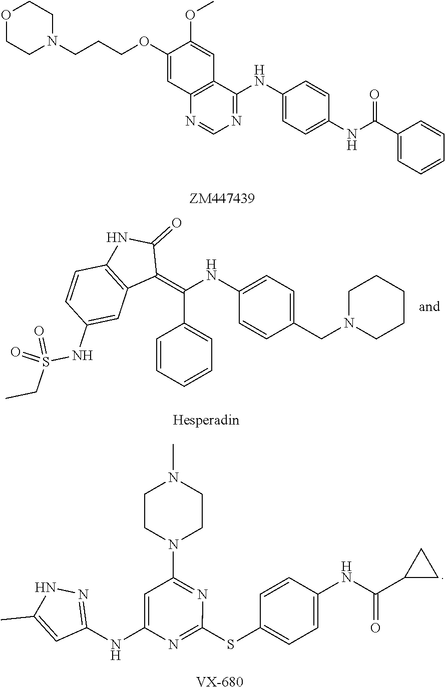

Compounds of the present invention or medicaments comprising the same can be prepared for administration using methodology well known in the pharmaceutical art. Examples of suitable pharmaceutical formulations and carriers are described in “Remington's Pharmaceutical Sciences” by E. W. Martin.

Suitable examples of the administration form of compounds of the present invention or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques.

Pharmaceutical compositions of the invention can be formulated so as to allow a compound according to the present invention to be bioavailable upon administration of the composition to an animal, preferably human. Compositions can take the form of one or more dosage units, where for example, a tablet can be a single dosage unit, and a container of a compound according to the present invention may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example, inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions according to the present invention, and pharmaceutically acceptable carriers are sterile. Water is a preferred carrier when the compounds according to the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The following examples further illustrate the invention. They should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

### Example 1—siRNA Screen to Identify Genes that Selectively Inhibited HORMAD1 Expressing Cells

A doxycycline-inducible HORMAD1 expression construct in which HORMAD1 expression could be induced in non-tumour retinal epithelial (RPE1) cells by exposure to doxycycline was developed to create an isogenic system to model the effect of HORMAD1 in cancers. Cells where exposed to doxycycline and an siRNA screen was carried out to identify genes which selectively inhibited HORMAD1 expressing cells.

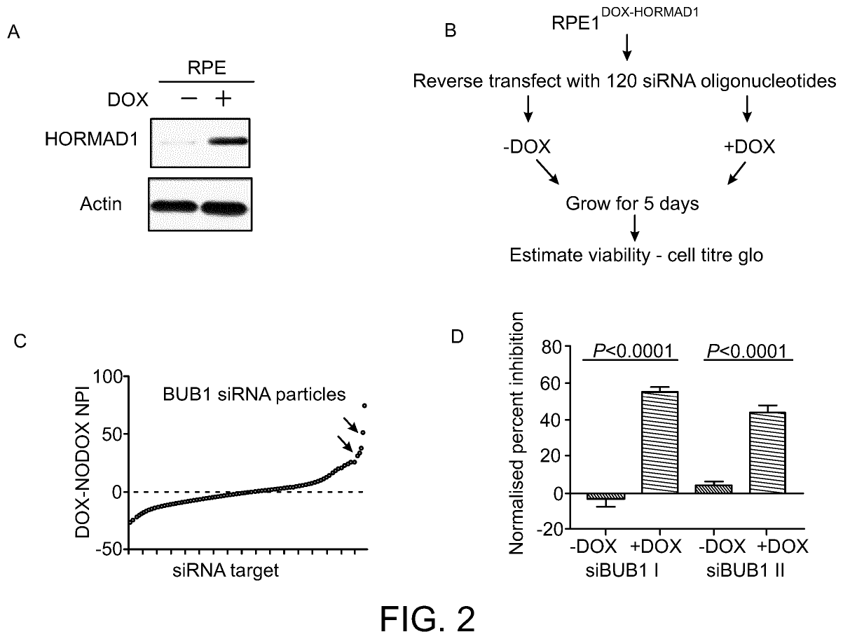

FIG. 2a shows the viability of the expression construct. RPE1 cells expressing a doxycycline-inducible HORMAD1 gene expression construct treated with doxycycline or vehicle for 3 days. Cell lysates analysed using SDS-PAGE and western blotting with anti-HORMAD1 and anti-ACTIN antibodies.

FIG. 2b shows a diagram illustrating the siRNA screen: RPE1 cells expressing a doxycycline-inducible HORMAD1 construct were treated with doxycycline or vehicle and reverse transfected with an siRNA screen targeting 30 genes (4 siRNA particles per gene). 5 days post HORMAD1 induction and siRNA transfection, cell viability was estimated using cell titre glo reagent. Cells were reverse transfected in a 384 well plate format with a siRNA library designed to target 30 proteins as described in Williamson et al. 2016.; twenty four hours after siRNA transfection, HORMAD1 expression was induced by exposing the cells to 500 ng/ml doxycycline. Cells were then continuously cultured for 5 days, at which point cell viability was assessed in each well by the use of a luminescent assay that detects cellular ATP (CellTitre Glo reagent).

Normalised percentage cell inhibition was calculated (normalised to non-targeting and PLK1 siRNA) for each siRNA used in the screen. FIG. 2c presents the results as the normalised percent inhibition (NPI) from doxycycline treated cells subtracted by the NPI of vehicle treated cells. Each dot represents the average from 5 replica plates for each siRNA oligonucleotide. The screen identified two different BUB1 siRNAs that selectively targeted RPE1 cells that expressed HORMAD1, but which did not profoundly affect the viability of HORMAD1-negative cells. The BUB1 siRNA particles are shown by arrow in FIG. 2c; and the HORMAD1-positive and—negative cell results are shown in FIG. 2d which is a bar chart depicting 5 replicates for the 2 BUB1 siRNA particles identified in siRNA screen (data analysed using Student's t-test, Bonferroni correction). It can therefore be concluded that HORMAD1 expression causes genetic addiction to BUB1.

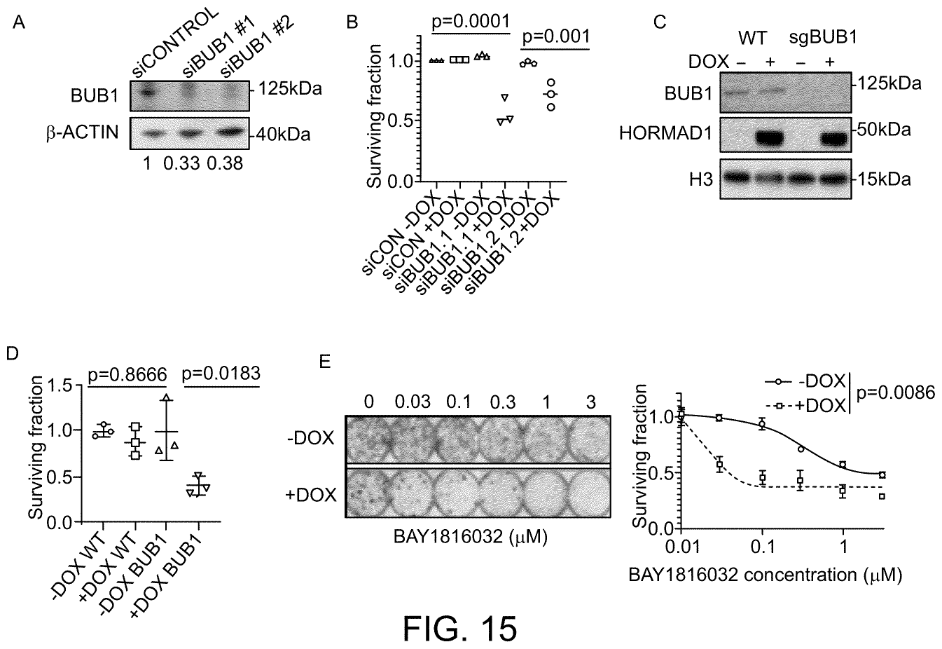

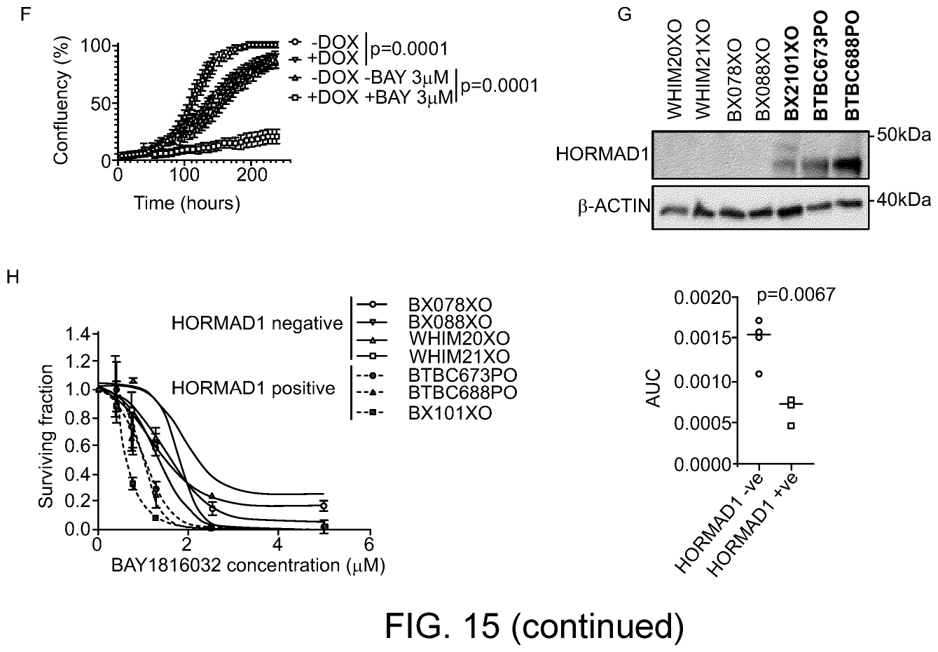

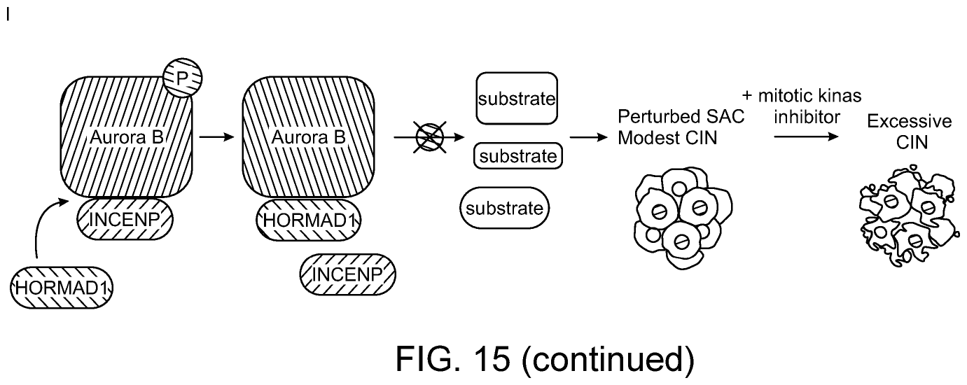

We then further exposed RPE1 cells to doxycycline to express HORMAD1, depleted BUB1 with two individual siRNAs, and estimated cellular viability after five days in culture. These experiments revealed a selective addiction to BUB1 in HORMAD1 expressing RPE1 cells (FIG. 15A-B). We validated these findings in RPE1 cells depleted of BUB1 using CRISPR-Cas9 gene editing (FIG. 15C-D). Importantly, these cells still express low levels of BUB1 protein that appears to be functionally important). These data indicate that HORMAD1 expression is synthetic lethal with BUB1 inhibition in somatic cells.

### Example 2—Effect of BUB1 Kinase Inhibitor, BAY-1816032 on HORMAD1 Expressing Cells

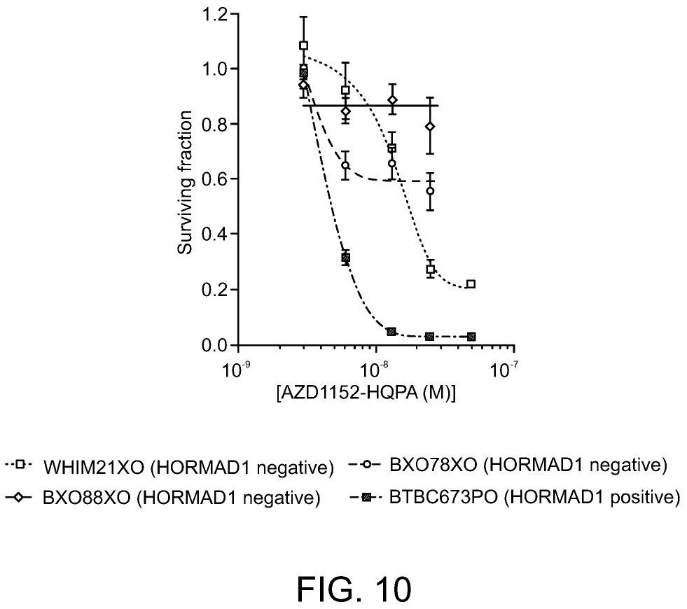

More recently, small molecule BUB1 inhibitors have been developed as potential cancer therapeutics with the intention of use in combination with microtubule interacting chemotherapies (Siemeister et al., 2019) but biomarker patient selection strategies that could direct the use of BUB1 inhibitors to maximum effect patient populations are not yet available. The effect of a small molecule BUB1 kinase inhibitor, BAY-1816032 was tested in the same cellular model. BAY-1816032 (CAS NO: 1891087-61-8) is known to inhibit BUB1 kinase activity by competitive inhibition of ATP binding (see, Siemeister et al., 2019, FIG. 10a).

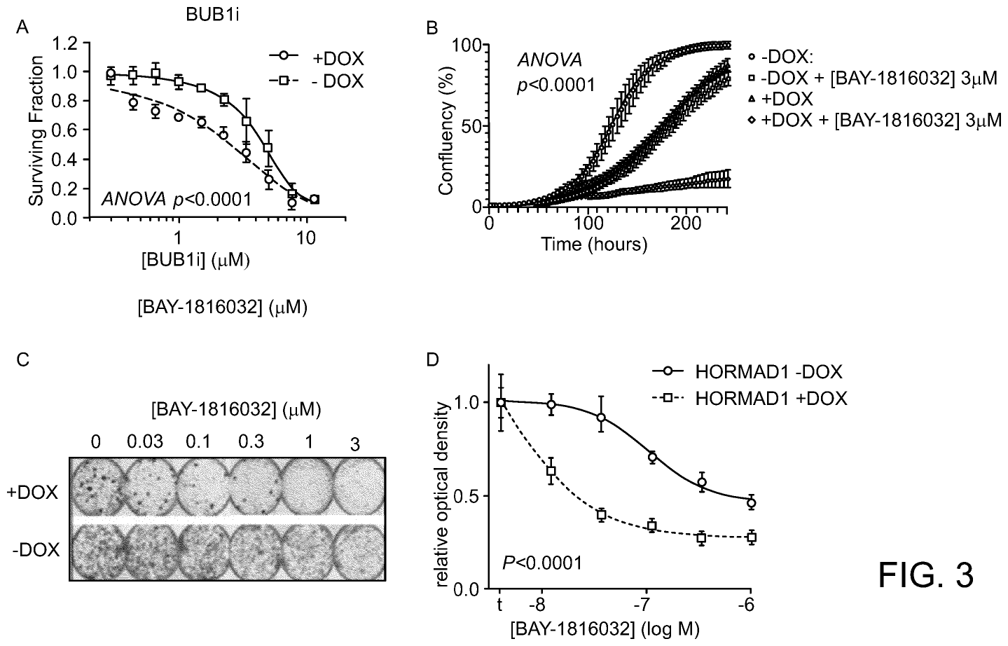

RPE1 cells expressing a doxycycline-inducible HORMAD1 construct were treated with doxycycline or vehicle. Following doxycycline induction, increasing doses of BUB1 inhibitor BAY-1816032 was added to the cells. Six days post HORMAD1 induction and 5 days post drug, cell viability was estimated using cell titre glo reagent. Surviving fractions were calculated (normalised to vehicle treated cells). The data is from four replicas, and analysed using 2-way ANOVA. The results are shown in FIG. 3a which show that HORMAD1 expressing RPE1 cells were more effectively inhibited by BUB1 inhibition than HORMAD1-negative RPE1 cells.

Next, following doxycycline exposure, we exposed RPE1 cells to 3 μm BAY-1816032 and used time-lapse imaging to measure cellular proliferation over 10 days. RPE1 cells expressing a doxycycline-inducible HORMAD1 construct were treated with doxycycline or vehicle. Following doxycycline induction, 3 μM BUB1 inhibitor, BAY-1816032, was added to cells. Using the incucyte software, phase contrast images of cells were captured every 4 hours for 240 hours and cell viability was estimated using confluency measurements. Data from four replicas were analysed using 2-way ANOVA. The results are shown in FIG. 3b which show that whilst RPE1 cells without HORMAD1 expression were able to grow to ˜85% confluency in the presence of a BUB1 inhibitor in this time period, HORMAD1 expressing RPE1 cells failed to proliferate and remained at around 15% confluency.

The clonogenic capacity of cells exposed to BAY-1816032 was also evaluated. RPE1 cells expressing a doxycycline-inducible HORMAD1 construct were treated with doxycycline or vehicle. Following doxycycline induction, increasing doses of BUB1 inhibitor BAY-1816032 was added to cells. 11 days post HORMAD1 induction and 10 days post drugging, cell viability was estimated by staining cells with SRB (left) and measuring SRB absorbance (right). Surviving fractions were calculated (normalised to

vehicle treated cells). Data is from three replicas, analysed using 2-way ANOVA. The results are shown in FIGS. 3c and d, which show that in the presence of HORMAD1 expression, the clonogenic capacity of RPE1 cells was reduced by 60% compared to that in RPE1 cells that did not express HORMAD1.

The results of these examples confirm that HORMAD1 creates a BUB1 dependency or synthetic lethality that can be targeted by small molecule inhibitors.

### Example 3—Efficacy of BUB1 Inhibitors on HORMAD1 Positive Cancer Cell-Lines

To confirm that endogenous HORMAD1 expression in cancer cells (as opposed to transgene-driven HORMAD1 expression) also causes BUB1 inhibitor sensitivity, increasing doses of BUB1 inhibitor was added to 3 HORMAD1-positive triple-negative breast cancer cell-lines (TNBC) and 2 HORMAD1-negative non-transformed breast cell-lines.

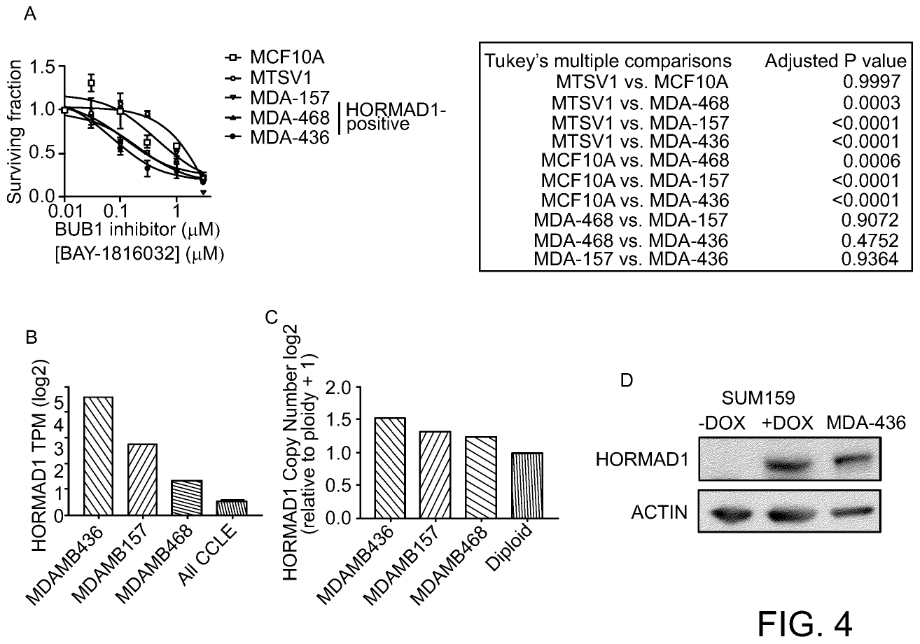

Three HORMAD1 positive TNBC cell-lines (MDA-157, MDA-468, MDA436) and two non-transformed breast cell-lines (MCF10A and MTSV1) were treated with increasing doses of BUB1 inhibitor BAY-1816032. 10-14 days post drugging, cell viability was estimated by staining cells with SRB and measuring absorbance. Surviving fractions were calculated (normalised to vehicle treated cells). Data is from 3 replicas, analysed using 2-way ANOVA. The results are shown in FIG. 4a, which reveal significant differences between all three HORMAD1-positive cell-lines, when compared with both HORMAD1-negative non-transformed cell-lines.

The expression and amplification of the HORMAD1 gene in all three triple-negative cell lines was confirmed using the cancer cell-line encyclopaedia dataset (Barretina et al., 2012, https://portals.broadinstitute.oro/ccle/pade?oene=HORMAD/). The results are reproduced as FIGS. 4b and 4c which show increased HORMAD1 mRNA expression in MDAMB-157, MDAMB-468 and MDAMB-436 (4b) and HORMAD1 copy number amplification in MDAMB-157, MDAMB-468 and MDAMB-436 (4c).

FIG. 4d shows the ability to detect HORMAD1 protein expression in MDAMB-436 cells. HORMAD1-negative SUM159 cells and SUM159 cells expressing HORMAD1 under doxycycline control were used as positive and negative controls, respectively. Cell lysates analysed using SDS-PAGE and western blotting with anti-HORMAD1 and anti-ACTIN antibodies.

### Example 4—Effects of BUB1 Inhibitor on HORMAD1-Positive Organoids in Isogenic Models

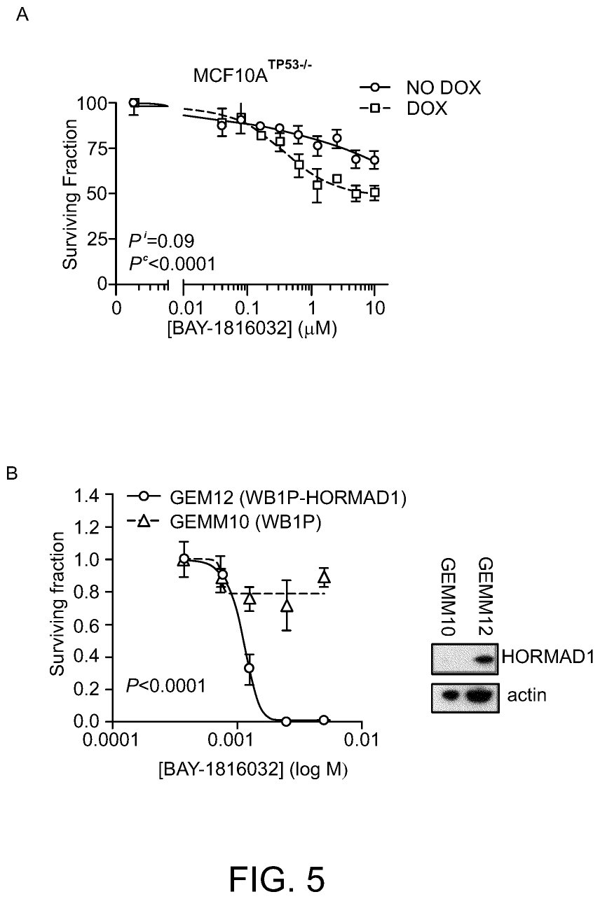

To demonstrate BUB1 inhibitor sensitivity of HORMAD1 expressing cells is replicated in more physiological relevant three-dimensional cultures, doxycycline-inducible HORMAD1 expression constructs were integrated into TP53-null MCF10A cells, which form spheroids when grown in semi-adherent cultures. TP53 null MCF10A cells expressing a doxycycline-inducible HORMAD1 construct were treated with doxycycline or vehicle. Cells were grown in 3D spheroid cultures. Following doxycycline induction, increasing doses of BUB1 inhibitor BAY-1816032 was added to the cells. 14 days post drugging, cell viability was estimated using cell titre glo reagent. Surviving fractions were calculated (normalised to vehicle treated cells). Data is from 3 replicas, analysed using 2-way ANOVA. It can be seen from the results in FIG. 5a that the growth of HORMAD1 expressing spheroids was more effectively inhibited by BUB1 inhibition than HORMAD1-negative spheroids.

In a further experiment, HORMAD1 was expressed within a genetically engineered mouse model of triple-negative breast cancer (original model published in Annunziato et al., 2019). Human HORMAD1 was expressed in the WB1P mouse (Annunziato et al., 2019) using a transgene and derived organoids from spontaneous tumours.

Genetically engineered mouse models were generated by conditionally deleting BRCA1 and P53 in breast epithelia (Liu et al., 2007). Human HORMAD1 fused to luciferase was conditionally expressed under a breast-specific promoter. Spontaneous tumours from these mice were isolated and grown as organoids, treated with increasing doses of BUB1 inhibitor, BAY-1816032. 14 days post drugging, cell viability was estimated using cell titre glo reagent. Surviving fractions were calculated (normalised to vehicle treated cells). Data from 3 replicas, analysed using 2-way ANOVA. It can be seen from FIG. 5b that the growth of HORMAD1-expressing organoids was more effectively inhibited by BAY-1816032 than HORMAD1-negative organoids.

It can be concluded from this example that HORMAD1 expression in 3D models of cancer leads to BUB1 inhibitor sensitivity.

### Example 5—Efficacy of BUB1 Inhibitors in HORMAD1-Positive Human Tumours

A further experiment was conducted to test the efficacy of BUB1 inhibitors to selectively kill HORMAD1-positive human tumours. Patient-derived organoids were grown from 3 HORMAD1-positive and 5 HORMAD1-negative tumours, from eight triple negative breast cancer patient-derived models (FIG. 15g). The organoids were exposed to increasing doses of the BUB1 inhibitor, BAY-1816032. 14 days post drugging, cell viability was estimated using cell titre glo reagent. Surviving fractions were calculated (normalised to vehicle treated cells). Data is from 3 replicas.

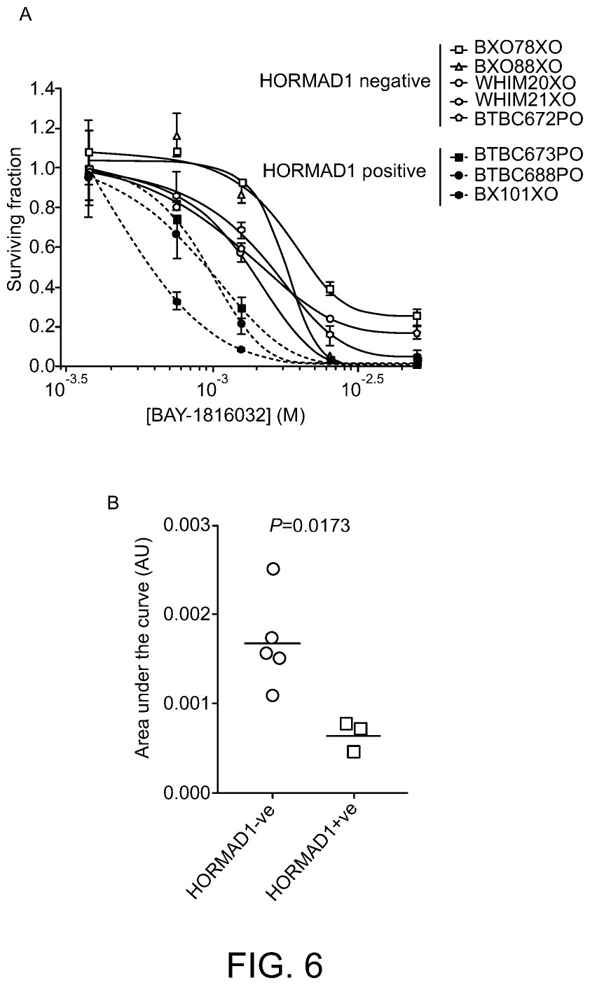

It can be seen from FIGS. 6a and 15h that the growth of HORMAD1-expressing patient samples were more effectively inhibited by BUB1 inhibition than HORMAD1-negative organoids. The area under the curve was calculated for each model and categorised based on HORMAD1 expression and the results (analysed using Student's t-test) are shown in FIGS. 6b and 15h. Expression of HORMAD1 was confirmed using RNA-seq, RNA-ish or western blotting and the results are shown in Table 1 below.

TABLE 1

| | RNA-seq | RNA-ish | Western blot |
|---|---|---|---|
| BX101XO | NT | + | NT |
| BTBC673 | NT | + | NT |
| BTBC688 | + | NT | + |
| BX078XO | − | − | NT |
| BX088 | − | − | NT |
| WHIM20-XO | − | NT | NT |
| WHIM21-XO | − | NT | NT |
| BTBC672PO | NT | NT | − |

(NT = not tested)

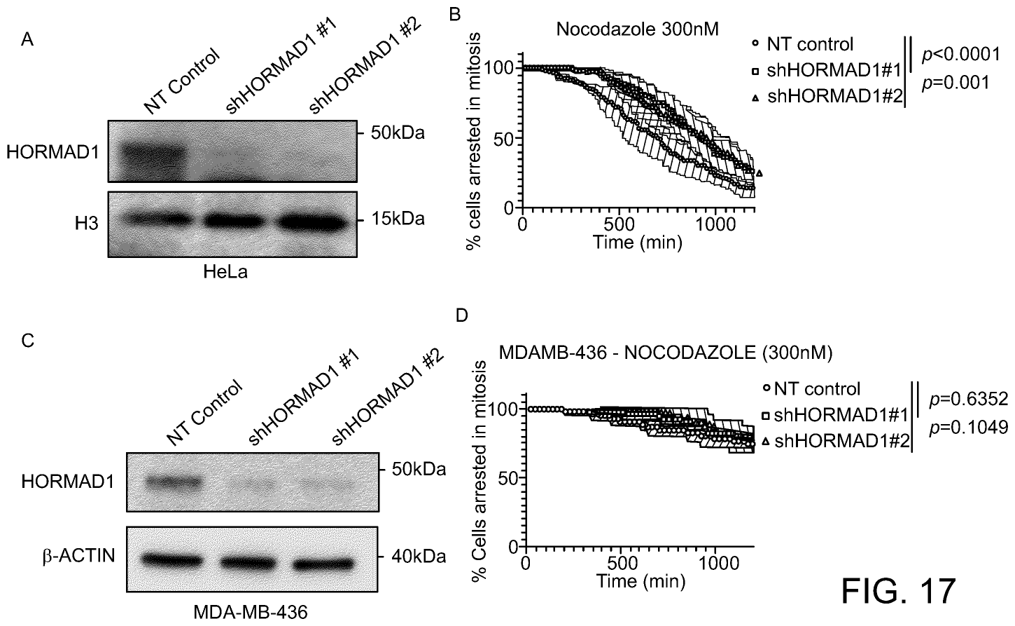

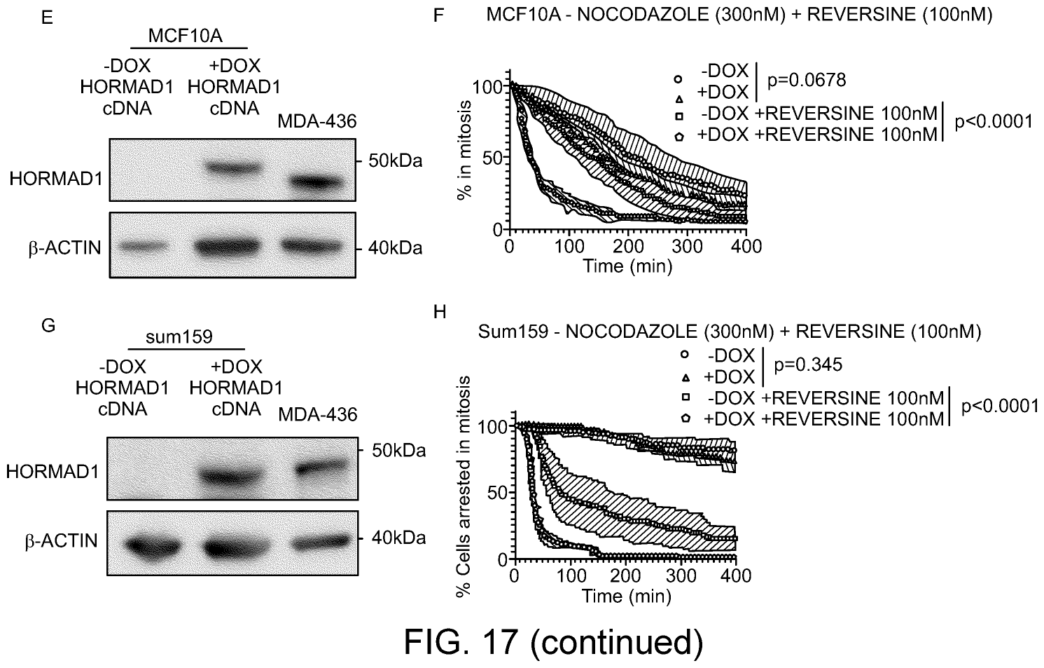

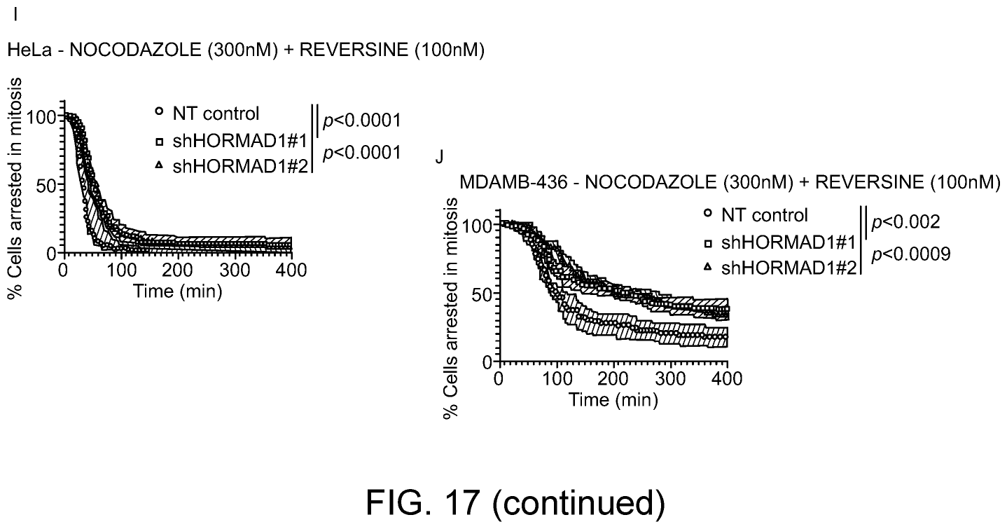

Taken all together, our data point towards a model in which HORMAD1 binds to Aurora B and inhibits its activity on the SAC and/or error correction, which leads to chromosomal instability. The addition of MPSI, Aurora B or BUB1 inhibitors thereby exposes HORMAD1 expressing cells to excessive chromosomal instability associated with loss of cell survival (FIG. 61). Given that HORMAD1 is not expressed in non-germline normal tissues but becomes expressed in 60% of TNBCs and a significant number of other high unmet need malignancies (FIG. 17), this indicates a selective dependence on several drug-able regulators of mitotic segregation.

### Example 6—HORMAD1 Sensitivity to MPSI Inhibitors

MPSI is a spindle assembly checkpoint kinase (Pachis & Kops, 2018). An experiment was conducted to confirm the sensitivity of HORMAD1 overexpressing cells with the MPSI inhibitor, BOS172722. BOS172722 is N2-(2-Ethoxy-4-(4-methyl-4 H-1,2,4-triazol-3-yl)phenyl)-6-methyl-N8-neopentylpyrido[3,4-d]pyrimidine-2,8-diamine, (CAS NO: 1578245-44-9), and inhibits MPSI kinase activity by competitive ATP binding inhibition (Woodward et al., 2018).

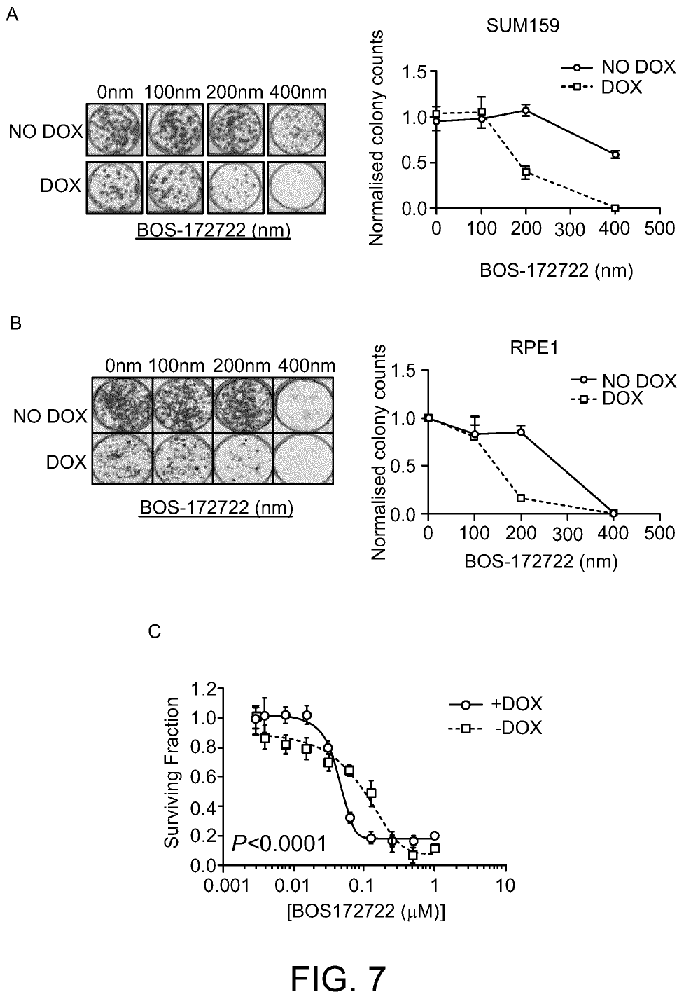

RPE1 and SUM159 cells expressing a doxycycline-inducible HORMAD1 construct were treated with doxycycline or vehicle. Following doxycycline induction, increasing doses of MPSI inhibitor, BOS172722, was added to the cells. 10-14 days after initiation of drug exposure, cell viability was estimated using clonogenic survival assays. Surviving fractions were calculated (normalised to vehicle treated cells). (FIGS. 7a, b and 14c and d). Data from 3 replicas was analysed using 2-way ANOVA. 5 days post drugging, cell viability was estimated using cell titre glo reagent. Surviving fractions were calculated (normalised to vehicle treated cells). Data from 4 replicas was analysed using 2-way ANOVA.

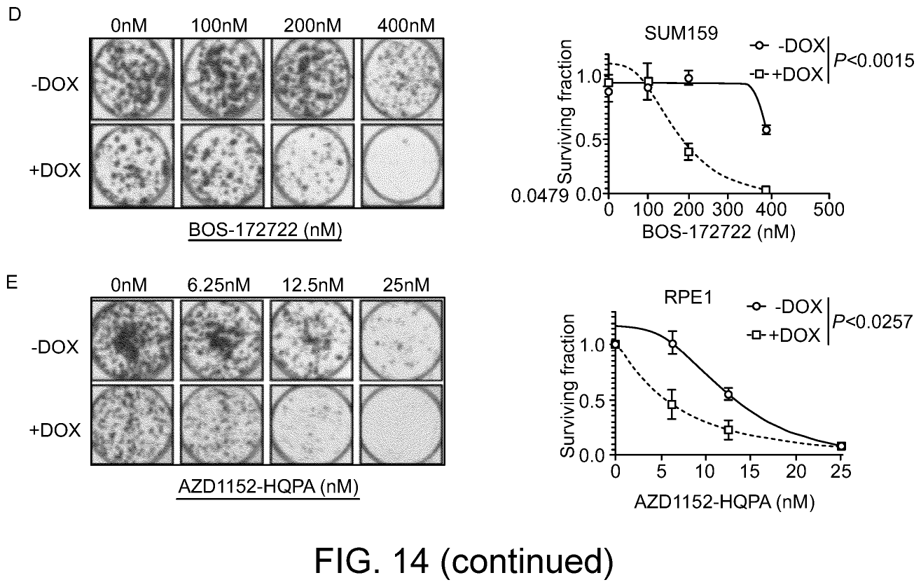

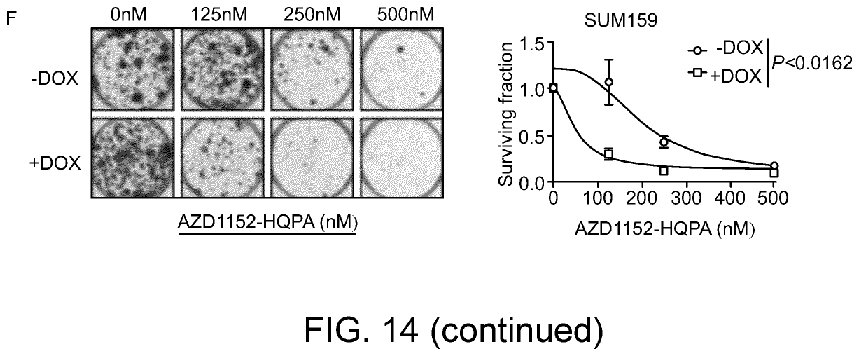

It can be seen from FIGS. 7a and 7b (and FIGS. 14c and d) that the survival of HORMAD1 expressing RPE1 and SUM159 cells was more effectively inhibited by MPSI inhibition when compared HORMAD1-negative RPE1 cells.

It can be seen from FIG. 7c that the growth of HORMAD1 expressing RPE1 cells was more effectively inhibited by MPSI inhibition when compared HORMAD1-negative RPE1 cells.

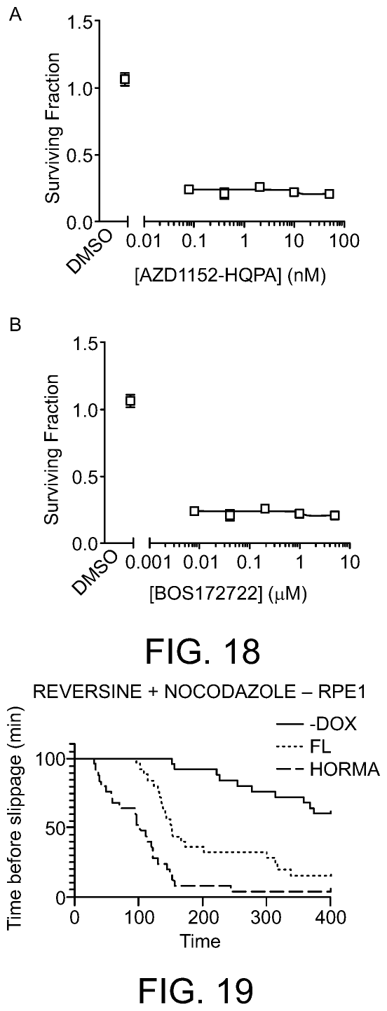

FIG. 18B shows that the HORMAD1 positive organoid KCL625 is highly sensitive to MPSI inhibition. It can be seen that treating organoids with the MPSI inhibitor BOS172722 for 10 days resulted in a reduced surviving fraction compared to DMSO treatment.

### Example 7—HORMAD1 Sensitivity to AURORA Kinase Inhibitors

Experiments were conducted to demonstrate that HORMAD1 overexpression also leads to sensitivity for AURORA kinase inhibitors, namely AURORA A and AURORA B.

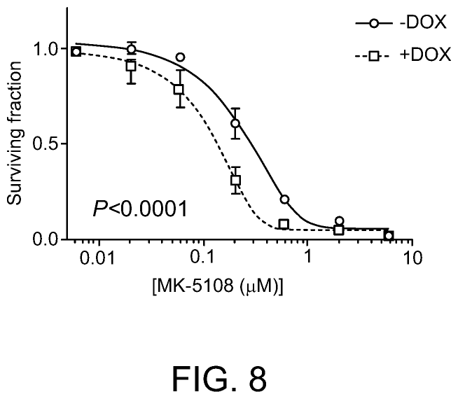

RPE1 cells expressing a doxycycline-inducible HORMAD1 construct were treated with doxycycline or vehicle. Following doxycycline induction, increasing doses of an AURORA A inhibitor, MK-5108, was added to cells. 5 days post drugging, cell viability was estimated using cell titre glo reagent. Surviving fractions were calculated (normalised to vehicle treated cells). Data from three replicas were analysed using 2-way ANOVA. The results are shown in FIG. 8 which show that the growth of HORMAD1 expressing RPE1 cells were more effectively inhibited by AURORA A inhibition when compared HORMAD1-negative RPE1 cells.

A further experiment was conducted to test an AURORA B kinase inhibitor, AZD1152-HQPA, using clonogenic survival assays.

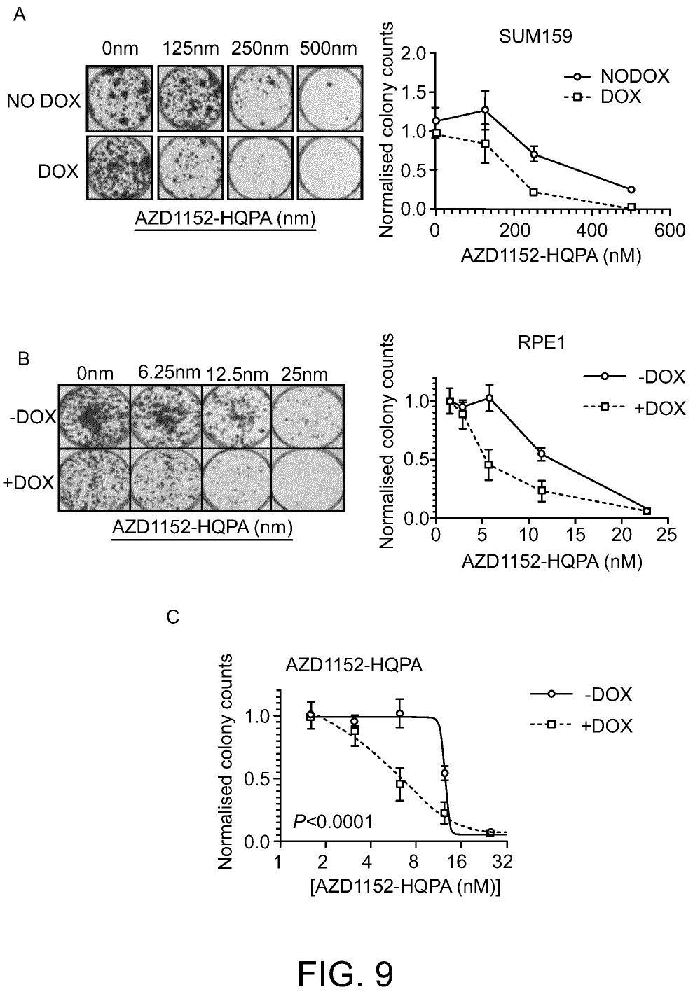

RPE1 and SUM159 cells expressing a doxycycline-inducible HORMAD1 construct were treated with doxycycline or vehicle. Following doxycycline induction, increasing doses of AURORA B inhibitor AZD1152-HQPA was added to SUM159 (FIG. 9a) and RPE1 (FIGS. 9b and c and FIGS. 14e and f) cells. The results are shown in FIG. 9. 9-13 days after HORMAD1 induction and 10-14 days after initiation of drug exposure, cell viability was estimated by staining cells with SRB and measuring clonogenic survival. Surviving fractions were calculated (normalised to vehicle treated cells). Data from 3 replicas were analysed using 2-way ANOVA.

These experiments revealed that the growth of HORMAD1 expressing RPE1 cells was more effectively inhibited by AURORA B inhibition when compared HORMAD1-negative RPE1 cells.

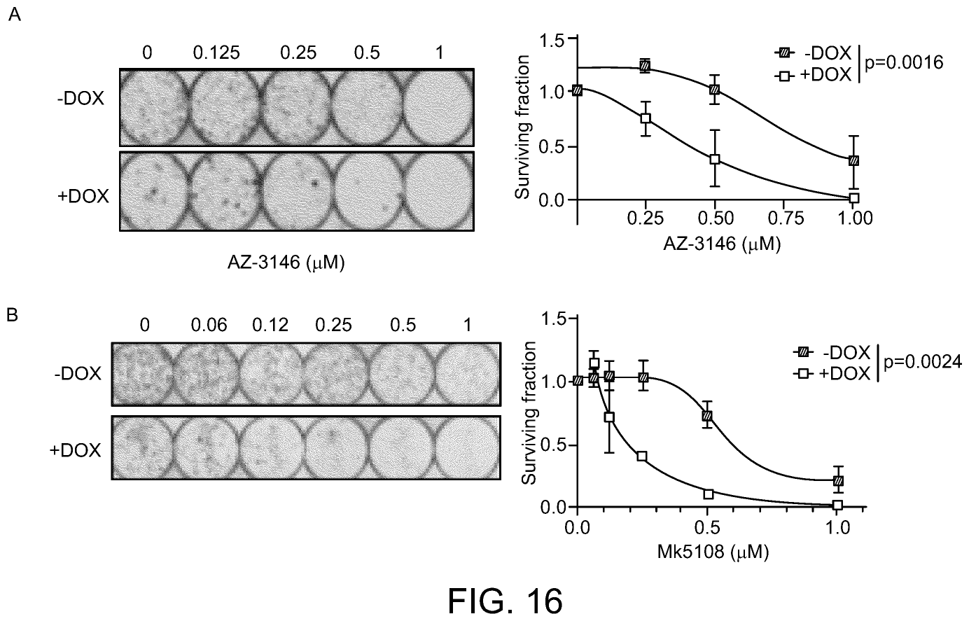

Accordingly, we have demonstrated that HORMAD1 expression enhances mitotic slippage induced by either MPSI or Aurora B inhibition, indicating that somatic cells expressing HORMAD1 would be hyper-dependent upon MPSI and residual Aurora B kinase activity for cell fitness, proliferation and clonal survival. We inhibited MPSI or Aurora B with their respective inhibitors, BOS172722 and AZD1152-HQPA, and measured cellular proliferation using Incucyte live cell microscopy. HORMAD1 expression resulted in a marked decrease in cellular growth when RPE1 cells were exposed to BOS172722 or AZD1152-HQPA (FIG. 14A-B). HORMAD1 also reduced clonogenic survival after MPSI or Aurora B inhibitor exposure measured by colony formation assays in both RPE1 cells and SUM159 cells (FIG. 14c-f). To ensure these were not compound specific effects we confirmed these findings with alternative MPSI and Aurora kinase inhibitors, AZ-3146 and MK-5108, respectively (FIG. 16). These data demonstrate that HORMAD1 expression exacerbates MPSI and Aurora B inhibitor sensitivity, consistent with the biological effects of HORMAD1 we demonstrate on mitotic slippage in the presence of these agents.

### Example 8—3D Organoid Experiments Using AURORA B Inhibitor AZD1152-HQPA

To further demonstrate the efficacy of AURORA B inhibitors to selectively kill HORMAD1-positive tumours, patient-derived organoids from one HORMAD1-positive (BTBC673PO) and three HORMAD1-negative (WHIM21XO, BXO88XO and BXO78XO) TNBC tumours were grown. The organoids were exposed to increasing doses of the AURORA B inhibitor, AZD1152-HQPA. 14 days post drugging, cell viability was estimated using cell titre glo reagent. Surviving fractions were calculated (normalised to vehicle treated cells) and data is from three replicas and analysed using 2-way ANOVA. HORMAD1 expression from patient organoids was assessed confirmed using RNA-seq, RNA-ish or western blotting as per Example 5. The results are shown in FIG. 10 and it can be seen that growth inhibition was more effective in HORMAD1 expressing organiods than HORMAD1-negative organoids.

Further to this, FIG. 18A shows that the HORMAD1 positive organoid KCL625 is highly sensitive to the AURORA B inhibitor AZD1152-HQPA. It can be seen that when treated with AZD1152-HQPA doses for 10 days the surviving fraction is much less than can be seen for DMSO treatment.

### Example 9—Spindle Assembly Checkpoint Defects and Mitotic Segregation Errors

BUB1, AURORA B, and MPSI share a common function of ensuring accurate chromosome segregation during mitosis. An experiment was conducted to investigate whether HORMAD1 perturbs this process.

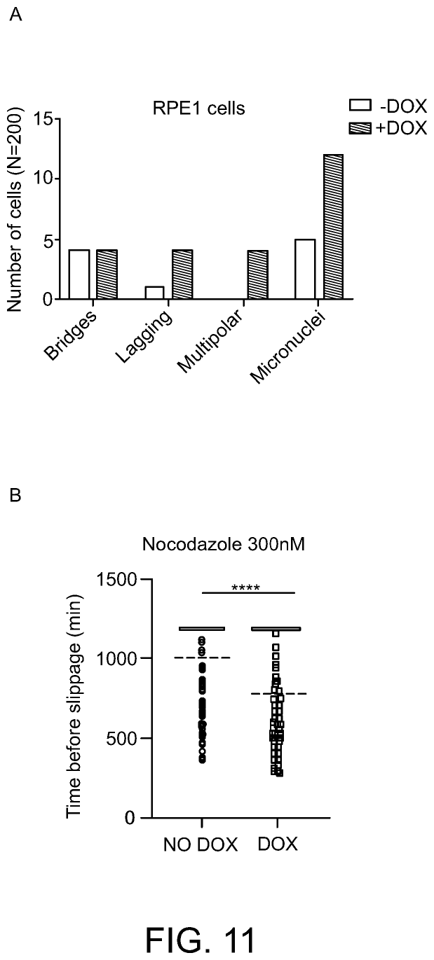

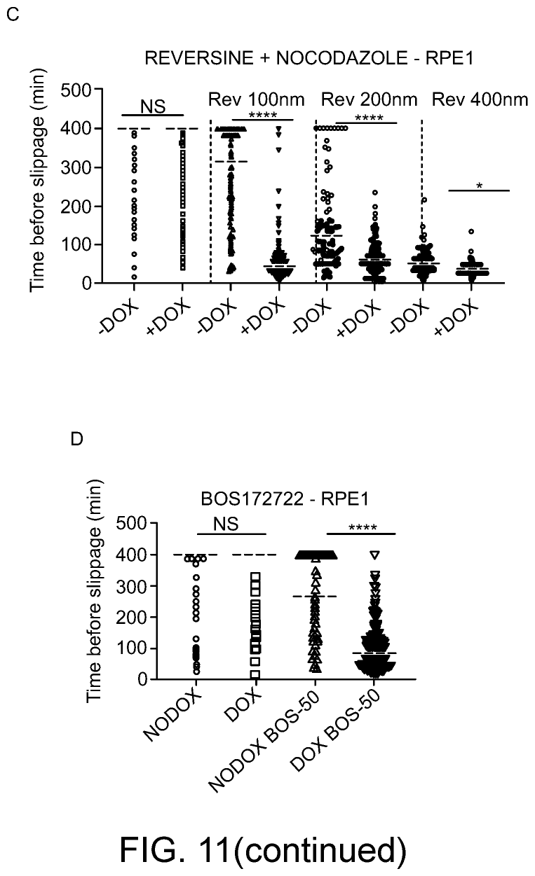

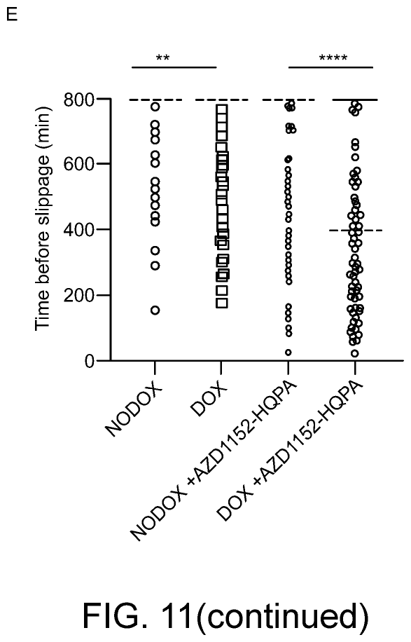

To test this, RPE1 cells expressing a doxycycline-inducible HORMAD1 construct were labelled with H2B-GFP and time-lapse fluorescence microscopy was used to assess segregation errors. Cells were exposed to doxycycline or vehicle for 3 days and segregation errors were quantified from 200 cells. The results are shown in FIG. 11a and it can be seen that HORMAD1 positive cells create more segregation errors, particularly lagging, multipolar and micronuclei errors.

Since BUB1, AURORA B, and MPSI are required for spindle assembly checkpoint, the potential for HORMAD1 to exacerbate spindle assembly checkpoint defects caused by inhibition of these mitotic kinases was investigated. Cells with a spindle assembly checkpoint defect rapidly exit mitosis, even when spindle fibres have not fully attached to centromeres. To test whether HORMAD1 induced a defect in this process, RPE1 cells, stably expressing a doxycycline-inducible HORMAD1 expression construct and labelled with H2B-GFP were exposed to doxycycline or vehicle for 3 days. Cells were exposed to high dose (300 nm) nocodazole in order to arrest cells in mitosis, before filming cells using time-lapse microscopy. The time taken for cells to exit mitosis via mitotic slippage was quantified from 100 cells. Data was analysed using a Student's t-test. The results are shown in FIG. 11b and it can be seen that only modest defects in the spindle assembly checkpoint were induced by HORMAD1.

The experiment was then repeated but a low dose of the toolbox MPSI inhibitor, reversine (2-(4-morpholinoanilino)-6-cyclohexylaminopurine) was added. Reversine has previously been shown to illuminate unidentified spindle assembly checkpoint defects (Raaijmakers & Medema, 2019). Thus, RPE1 cells, stably expressing a doxycycline-inducible HORMAD1 expression construct, were exposed to doxycycline or vehicle for 3 days. Cells were exposed to 300 nm nocodazole for 8 hours in order to arrest cells in mitosis. MPSI inhibitor (Reversine) (100 nm) was added immediately before filming in order to induce mitotic slippage. The time taken for cells to exit mitosis via mitotic slippage was quantified from 100 cells. Data was analysed using a one-way ANOVA. The results are shown in FIG. 11c and it can be seen that HORMAD1 induction caused a clear and obvious spindle assembly checkpoint defect, as measured by rapid mitotic exit after treatment with reversine. This experiment was repeated but with the clinically relevant MPSI inhibitor, BOS172722 (at 50 nm) and the AURKB inhibitor (AZD1152-HQPA). Very similar results were seen as shown in FIGS. 11d and 11e.

Through this experiment, it can be seen that agents that modulate mitotic processes exploit spindle assembly checkpoint defects and mitotic segregation errors in HORMAD1 overexpressing cancers.

Thus, identification of spindle assembly checkpoint defects, whether caused by HORMAD1 or otherwise, can be used as a biomarker for agents that modulate mitotic processes. Exemplary spindle assembly checkpoint defects include segregation errors, particularly lagging, multipolar and/or micronuclei errors.

Examples 1 to 9 demonstrate that elevated HORMAD1 expression causes profound sensitivity to agents that modulate mitotic processes, effects that can be exploited by the use of clinical, drug like, inhibitors. Exemplary inhibitors include BUB1 inhibitors, MPSI inhibitors and AURORA kinase inhibitors including AURORA A kinase inhibitors and AURORA B kinase inhibitors. Having demonstrated the effectiveness of agents that modulate mitotic processes in the treatment of cancers that overexpress HORMAD1, experiments were conducted to evaluate whether this effect is replicated in combination with other anticancer agents.

The effect of the HORMA domain alone versus the entre HORMAD1 protein was also investigated. FIG. 19 demonstrates that the expression of the HORMA domain of the HORMAD1 protein impairs mitotic arrest. RPE1 cells expressing a doxycycline-inducible full length or HORMA domain only expression construct were exposed to doxycycline or vehicle for three days, at which point cells were exposed to 300 nM nocodazole for 6 hours, and then 100 nM reversine, before being filmed every 2 minutes using time-lapse microscopy. The time individual cells remained arrested in mitosis was quantified and presented as the % of cells arrested in mitosis at each time point. Data reflects a total of 150 cells from 3 biological repeats and was analysed using Mantel-Cox test with Bonferonni correction. Here is clearly seen that the time before slippage in cells expressing only the HORMA domain is reduced.

### Example 10—Combination of Agents that Modulate Mitotic Processes and a Microtubule Inhibitor or Disrupter

Eribulin is a chemotherapeutic agent commonly used in the treatment of cancer and with a specific role in advanced breast cancer including the Triple Negative subtype (see Kaufman 2015 and Cortes 2011). Eribulin mesylate is a microtubule disrupter as a result of inhibition microtubule polymerisation. It prevents the microtubules from growing but does not affect the shortening of the microtubules in mitosis, making the tubulin non-functional. It disrupts mitotic spindles, and this causes cells to arrest in the M-phase of the cell-cycle, which leads to cell death as a result of mitotic blockage.

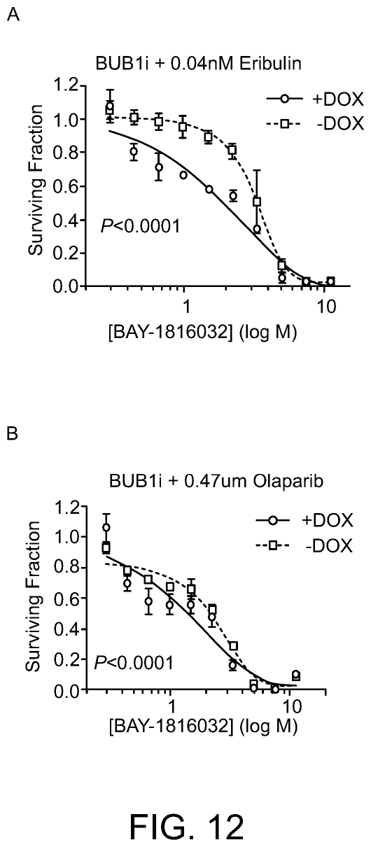

An experiment was conducted to confirm whether HORMAD1-driven BUB1 inhibitor sensitivity would persist in cells that were treated with eribulin. RPE1 cells expressing a doxycycline-inducible HORMAD1 construct were treated with doxycycline or vehicle in order to induce HORMAD1 expression. Increasing doses of BUB1 inhibitor BAY-1816032 was added alongside 0.4 nm eribulin. Using cell titre glo as a measure of cell viability, surviving fractions were calculated (normalised to eribulin alone). Data is from 4 replicas and analysed using 2-way ANOVA. The results are shown in FIG. 12a and it can be seen that growth of HORMAD1 expressing RPE1 cells was more effectively inhibited by BUB1 inhibition when compared HORMAD1-negative RPE1 cells.

### Example 11—Combination of Agents that Modulate Mitotic Processes and a PARP Inhibitor

An experiment was conducted to confirm whether HORMAD1 driven sensitivity is retained in the presence of a second agent with a different mechanism of action. PARP inhibitors are small molecule agents currently under investigation in triple-negative breast cancers.

RPE1 cells expressing a doxycycline-inducible HORMAD1 construct were treated with doxycycline or vehicle. Cells were then treated with increasing doses of the BUB1 inhibitor, BAY-1816032, alongside 0.47 μm of the PARP1 inhibitor olaparib. Using cell titre glo as a measure of cell viability, surviving fractions were calculated (normalised to olaparib alone). Data is from four replicas and analysed using 2-way ANOVA. The results are shown in FIG. 12b and it can be seen that the growth of HORMAD1 expressing RPE1 cells was more effectively inhibited by BUB1 inhibition when compared HORMAD1-negative RPE1 cells.

Examples 10 and 11 demonstrate that HORMAD1-driven sensitivity to agents that modulate mitotic processes persists in the presence of additional therapeutic agents and that the HORMAD1/agent that modulates mitotic processes lethality could be exploited not only when these agents are used as single agents but also when used in combination with other treatment approaches.

In conclusion, the present invention has identified that HORMAD1 gene amplification, mRNA expression, or protein expression can be used as a cancer biomarker; and that this can be used to target the use of agents that modulate mitotic processes.

The present invention has shown for the first time that elevated HORMAD1 expression causes profound sensitivity to agents that modulate mitotic processes, effects that can be exploited by the use of clinical, drug like, inhibitors. Exemplary inhibitors include BUB1 inhibitors, MPSI inhibitors and AURORA kinase inhibitors including AURORA A kinase inhibitors and AURORA B kinase inhibitors.

Further, since HORMAD1 can be readily detected in tumour samples, this data provides a pre-clinical rationale for the use of HORMAD1 expression as a predictive biomarker of sensitivity to agents that modulate mitotic processes, including inhibitors of either BUB1, MPSI or AURORA kinases including AURORA A or AURORA B.

It has further been identified that spindle assembly checkpoint defects, caused by HORMAD1 or otherwise, can be used as a biomarker for agents that modulate mitotic processes.

Finally, it has been confirmed that HORMAD1-d riven sensitivity to agents that modulate mitotic processes is maintained in combination therapies.

### REFERENCES

Anderhub, S. J., G. W. Mak, M. D. Gurden, A. Faisal, K. Drosopoulos, K. Walsh, H. L. Woodward, et al. “High Proliferation Rate and a Compromised Spindle Assembly Checkpoint Confers Sensitivity to the Mps1 Inhibitor Bos172722 in Triple-Negative Breast Cancers.” [In eng]. Mol Cancer Ther 18, no. 10 (Oct 2019): 1696-707.
Annunziato, S., J. R. de Ruiter, L. Henneman, C. S. Brambillasca, C. Lutz, F. Vaillant, F. Ferrante, et al. “Comparative Oncogenomics Identifies Combinations of Driver Genes and Drug Targets in Brca1-Mutated Breast Cancer.” [In eng]. Nat Commun 10, no. 1 (Jan. 23, 2019): 397.
Baron, A. P., C. von Schubert, F. Cubizolles, G. Siemeister, M. Hitchcock, A. Mengel, J. Schroder, et al. “Probing the Catalytic Functions of Bub1 Kinase Using the Small Molecule Inhibitors Bay-320 and Bay-524.” [In eng]. Elife 5 (Feb. 17, 2016).
Barretina, J., G. Caponigro, N. Stransky, K. Venkatesan, A. A. Margolin, S. Kim, C. J. Wilson, et al. “The Cancer Cell Line Encyclopedia Enables Predictive Modelling of Anticancer Drug Sensitivity.” [In eng]. Nature 483, no. 7391 (Mar. 28, 2012): 603-7.
Bolanos-Garcia, V. M., and T. L. Blundell. “Bub1 and Bubr1: Multifaceted Kinases of the Cell Cycle.” [In eng]. Trends Biochem Sci 36, no. 3 (Mar. 2011): 141-50.
Carofiglio, F., E. Sleddens-Linkels, E. Wassenaar, A. Inagaki, W. A. van Cappellen, J. A. Grootegoed, A. Toth, and W. M. Baarends. “Repair of Exogenous DNA Double-Strand Breaks Promotes Chromosome Synapsis in Spo11-Mutant Mouse Meiocytes, and Is Altered in the Absence of Hormad1.” [In eng]. DNA Repair (Amst) 63 (Mar. 2018): 25-38.
Chen, Y. T., C. A. Venditti, G. Theiler, B. J. Stevenson, C. Iseli, A. O. Gure, C. V. Jongeneel, L. J. Old, and A. J. Simpson. “Identification of Ct46/HORMAD1, an Immunogenic Cancer/Testis Antigen Encoding a Putative Meiosis-Related Protein.” [In eng]. Cancer Immun 5 (Jul. 7, 2005): 9.
Kawashima, S. A., Y. Yamagishi, T. Honda, K. Ishiguro, and Y. Watanabe. “Phosphorylation of H2a by Bub1 Prevents Chromosomal Instability through Localizing Shugoshin.” [In eng]. Science 327, no. 5962 (Jan. 8, 2010): 172-7.
Kogo, H., M. Tsutsumi, T. Ohye, H. Inagaki, T. Abe, and H. Kurahashi. “Hormad1-Dependent Checkpoint/Surveillance Mechanism Eliminates Asynaptic Oocytes.” [In eng]. Genes Cells 17, no. 6 (Jun. 2012): 439-54.
Liu, X., H. Holstege, H. van der Gulden, M. Treur-Mulder, J. Zevenhoven, A. Velds, R. M. Kerkhoven, et al. “Somatic Loss of Brca1 and P53 in Mice Induces Mammary Tumors with Features of Human Brca1-Mutated Basal-Like Breast Cancer.” [In eng]. Proc Natl Acad Sci USA 104, no. 29 (Jul. 17, 2007): 12111-6.
Mortlock, A. A., K. M. Foote, N. M. Heron, F. H. Jung, G. Pasquet, J. J. Lohmann, N. Wenn, et al. “Discovery, Synthesis, and in Vivo Activity of a New Class of Pyrazoloquinazolines as Selective Inhibitors of Aurora B Kinase.” [In eng]. J Med Chem 50, no. 9 (May 3, 2007): 2213-24.
Musacchio, A. “The Molecular Biology of Spindle Assembly Checkpoint Signaling Dynamics.” [In eng]. Curr Biol 25, no. 20 (Oct. 19, 2015): R1002-18.
Oser, M. G., R. Fonseca, A. A. Chakraborty, R. Brough, A. Spektor, R. B. Jennings, A. Flaifel, et al. “Cells Lacking the Rb1 Tumor Suppressor Gene Are Hyperdependent on Aurora B Kinase for Survival.” [In eng]. Cancer Discov 9, no. 2 (Feb. 2019): 230-47.
Overlack, K., I. Primorac, M. Vleugel, V. Krenn, S. Maffini, I. Hoffmann, G. J. Kops, and A. Musacchio. “A Molecular Basis for the Differential Roles of Bub1 and Bubr1 in the Spindle Assembly Checkpoint.” [In eng]. Elife 4 (Jan. 22, 2015): e05269.
Pachis, S. T., and Gjpl Kops. “Leader of the Sac: Molecular Mechanisms of Mps1/Ttk Regulation in Mitosis.” [In eng]. Open Biol 8, no. 8 (Aug 2018).
Primorac, I., J. R. Weir, E. Chiroli, F. Gross, I. Hoffmann, S. van Gerwen, A. Ciliberto, and A. Musacchio. “Bub3 Reads Phosphorylated Melt Repeats to Promote Spindle Assembly Checkpoint Signaling.” [In eng]. Elife 2 (Sep. 24, 2013): e01030.
Qiao, H., Hbdp Rao, Y. Yun, S. Sandhu, J. H. Fong, M. Sapre, M. Nguyen, et al. “Impeding DNA Break Repair Enables Oocyte Quality Control.” [In eng]. Mol Cell 72, no. 2 (Oct. 18, 2018): 211-21.e3.
Raaijmakers, J. A., and R. H. Medema. “Killing a Zombie: A Full Deletion of the Bub1 Gene in Hap1 Cells.” [In eng]. Embo j 38, no. 22 (Nov. 15, 2019): e102423.
Ricke, R. M., K. B. Jeganathan, L. Malureanu, A. M. Harrison, and J. M. van Deursen. “Bub1 Kinase Activity Drives Error Correction and Mitotic Checkpoint Control but Not Tumor Suppression.” [In eng]. J Cell Biol 199, no. 6 (Dec. 10, 2012): 931-49.
Rosenberg, S. C., and K. D. Corbett. “The Multifaceted Roles of the Horma Domain in Cellular Signaling.” [In eng]. J Cell Biol 211, no. 4 (Nov. 23, 2015): 745-55.
Shimomura, T., S. Hasako, Y. Nakatsuru, T. Mita, K. Ichikawa, T. Kodera, T. Sakai, et al. “Mk-5108, a Highly Selective Aurora-a Kinase Inhibitor, Shows Antitumor Activity Alone and in Combination with Docetaxel.” [In eng]. Mol Cancer Ther 9, no. 1 (Jan. 2010): 157-66.
Siemeister, G., A. Mengel, A. E. Fernandez-Montalvan, W. Bone, J. Schroder, S. Zitzmann-Kolbe, H. Briem, et al. “Inhibition of Bub1 Kinase by Bay 1816032 Sensitizes Tumor Cells toward Taxanes, Atr, and Parp Inhibitors in Vitro and in Vivo.” [In eng]. Clin Cancer Res 25, no. 4 (Feb. 15, 2019): 1404-14.
Uhlen, M., L. Fagerberg, B. M. Hallstrom, C. Lindskog, P. Oksvold, A. Mardinoglu, A. Sivertsson, et al. “Proteomics. Tissue-Based Map of the Human Proteome.” [In eng]. Science 347, no. 6220 (Jan. 23, 2015): 1260419.
Uhlen, M., C. Zhang, S. Lee, E. Sjostedt, L. Fagerberg, G. Bidkhori, R. Benfeitas, et al. “A Pathology Atlas of the Human Cancer Transcriptome.” [In eng]. Science 357, no. 6352 (Aug. 18, 2017).
Wang, F., N. P. Ulyanova, M. S. van der Waal, D. Patnaik, S. M. Lens, and J. M. Higgins. “A Positive Feedback Loop Involving Haspin and Aurora B Promotes Cpc Accumulation at Centromeres in Mitosis.” [In eng]. Curr Biol 21, no. 12 (Jun. 21, 2011): 1061-9.
Watkins, J., D. Weekes, V. Shah, P. Gazinska, S. Joshi, B. Sidhu, C. Gillett, et al. “Genomic Complexity Profiling Reveals That HORMAD1 Overexpression Contributes to Homologous Recombination Deficiency in Triple-Negative Breast Cancers.” [In eng]. Cancer Discov 5, no. 5 (May 2015): 488-505.
Wilkinson, R. W., R. Odedra, S. P. Heaton, S. R. Wedge, N. J. Keen, C. Crafter, J. R. Foster, et al. “Azd1152, a Selective Inhibitor of Aurora B Kinase, Inhibits Human Tumor Xenograft Growth by Inducing Apoptosis.” [In eng]. Clin Cancer Res 13, no. 12 (Jun. 15, 2007): 3682-8.
Willems, E., M. Dedobbeleer, M. Digregorio, A. Lombard, P. N. Lumapat, and B. Rogister. “The Functional Diversity of Aurora Kinases: A Comprehensive Review.” [In eng]. Cell Div 13 (2018): 7.
Woodward, H. L., P. Innocenti, K. J. Cheung, A. Hayes, J. Roberts, A. T. Henley, A. Faisal, et al. “Introduction of a Methyl Group Curbs Metabolism of Pyrido[3,4-D]Pyrimidine Monopolar Spindle 1 (Mps1) Inhibitors and Enables the Discovery of the Phase 1 Clinical Candidate N(2)-(2-Ethoxy-4-(4-Methyl-4 H-1,2,4-Triazol-3-YI)Phenyl)-6-Methyl-N(8)-Neopentylpyrido[3,4-D]Pyrimidine-2,8-Diamine (Bos172722).” [In eng]. J Med Chem 61, no. 18 (Sep. 27, 2018): 8226-40.
Yang, J., T. Ikezoe, C. Nishioka, T. Tasaka, A. Taniguchi, Y. Kuwayama, N. Komatsu, et al. “Azd1152, a Novel and Selective Aurora B Kinase Inhibitor, Induces Growth Arrest, Apoptosis, and Sensitization for Tubulin Depolymerizing Agent or Topoisomerase li Inhibitor in Human Acute Leukemia Cells in Vitro and in Vivo.” [In eng]. Blood 110, no. 6 (Sep. 15, 2007): 2034-40.
Kaufman et al, “Phase III open-label randomized study of eribulin mesylate versus capecitabine in patients with locally advanced or metastatic breast cancer previously treated with an anthracycline and a taxane” J Clin Oncol. 2015 Feb. 20; 33(6):594-601
Cortes et al, “Eribulin monotherapy versus treatment of physician's choice in patients with metastatic breast cancer (EMBRACE): a phase 3 open-label randomised study” Lancet. 2011 Mar. 12; 377(9769):914-2
   https://clinicaltrials.qov/ct2/show/NCT03330847
Williamson C T, Miller R, Pemberton H N, Jones S E, Campbell J, Konde A, Badham N, Rafiq R, Brough R, Gulati A, Ryan C J, Francis J, Vermulen P B, Reynolds A R, Reaper P M, Pollard J R, Ashworth A, Lord C J. ATR inhibitors as a synthetic lethal therapy for tumours deficient in ARID1A. Nat Commun. 2016 Dec. 13; 7:13837. doi: 10.1038/ncomms13837. PMID: 27958275; PMCID: PMC5159945.

1. An agent that modulates mitotic processes for use in a method of treating a patient with cancer; said treatment comprising:
a) determining whether the cancer expresses HORMAD1; and, if so
b) administering to said patient an agent that modulates mitotic processes.

2. An agent that modulates mitotic processes for use in a method of treating a patient with cancer; said treatment comprising:
a) determining whether a test sample from the patient expresses HORMAD1; and, if so
b) administering to said patient an agent that modulates mitotic processes.

3. The agent for use according to claim 1 or claim 2, wherein the cancer is a HORMAD1 positive cancer selected from breast cancers, such as triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

4. An agent that modulates mitotic processes for use in the treatment of a HORMAD1 positive cancer, wherein preferably the cancer is selected from breast cancers, such as triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

5. The agent for use according to any one of claims 1 to 4, wherein the agent that modulates mitotic processes is a BUB1 inhibitor.

6. The agent for use according to any one of claims 1 to 4, wherein the agent that modulates mitotic processes is an MPSI inhibitor.

7. The agent for use according to any one of claims 1 to 4, wherein the agent that modulates mitotic processes is an AURORA kinase inhibitor.

8. The agent for use according to any one of claims 1 to 4, wherein the agent that modulates mitotic processes is an AURORA A kinase inhibitor.

9. The agent for use according to any one of claims 1 to 4, wherein the agent that modulates mitotic processes is an AURORA B kinase inhibitor.

10. The agent for use according to any one of claims 1 to 4, wherein the agent that modulates mitotic processes is a MASTL inhibitor.

11. The agent for use according to any one of claims 1 to 4, wherein the agent that modulates mitotic processes is a PLK1 inhibitor.

12. The agent for use according to any one of claims 1 to 4, wherein the agent that modulates mitotic processes is an inhibitor of cytokinesis.

13. The agent for use according to any preceding claim, wherein the agent is administered in combination with a further agent, wherein the further agent is preferably a microtubule disrupter.

14. A method of treating a patient with cancer, said method comprising:
a) determining whether said cancer expresses HORMAD1; and, if so
b) administering to said patient an agent that modulates mitotic processes.

15. A method of treating a patient with cancer, said method comprising:
a) determining whether a test sample from said patient expresses HORMAD1; and, if so
b) administering to said patient an agent that modulates mitotic processes.

16. An in-vitro method for identifying an individual with cancer having suitability for treatment with an agent that modulates mitotic processes, said method comprising determining whether a cell sample from said individual expresses HORMAD1.

17. An in-vitro method for identifying an individual with cancer having suitability for treatment with an agent that modulates mitotic processes; said method comprising identifying whether said cancer cells have spindle assembly checkpoint defects during mitosis; wherein the presence of spindle assembly checkpoint defects indicates sensitivity to an agent that modulates mitotic processes.

18. The method of any of claims 14 to 17, wherein the agent is administered in combination with a further agent, wherein the further agent is preferably a microtubule disrupter, microtubule stabiliser or DNA replication arresting agent.
